(19) United States
(12) Patent Application Publication
(10) Pub. No.: US 2024/0347149 A1
(43) Pub. Date: Oct. 17, 2024

(54) HEALTHCARE MANAGEMENT SYSTEM AND METHOD

(71) Applicant: STREAMLINE FLOW INC., Madison, WI (US)

(72) Inventors: Amanda Schleede, St. Charles, IL (US); Kelly Ayala, Whitefish Bay, WI (US); Shannon Hattenhauer, Madison, WI (US); Igor Soloshenko, Georgetown, TX (US); Paul Robke, Austin, TX (US)

(73) Assignee: STREAMLINE FLOW INC., Madison, WI (US)

(21) Appl. No.: 18/134,005

(22) Filed: Apr. 12, 2023

(51) Int. Cl. G16H 10/60

(52) U.S. Cl. CPC G16H 10/60

(57) ABSTRACT

A patient-centered healthcare management system with a provider dashboard interface and a patient application interface. The provider dashboard and patient application display synchronized data about patient care plans for each of a patient's diagnoses, including a next step and a risk level status for the next step. The risk level status is initially selected based on the provider's assessment of the risk that the next step will not be completed by an agreed-upon time. The risk level status is automatically elevated to a next level when the agreed-upon time passes. The provider dashboard includes a patient table that is sortable by a selected patient data value, facilitating multi-selection of patients for batch operations. The patient app receives automated reminders at preset times based on the patient's care plan, including guided self-advocacy prompts timed to occur during scheduled appointments or based on next step due dates.

### CROSS-REFERENCE

None.

### FIELD OF THE DISCLOSURE

The present disclosure relates to computer-implemented healthcare management systems and methods. More particularly, it relates to a healthcare patient tracking software platform including backend healthcare provider access via a provider dashboard user interface and patient access via a mobile app user interface.

### SUMMARY

According to an aspect of the disclosure, a computer-implemented healthcare management system comprises a processor, a computer readable memory device operatively connected to the processor, a provider input device, a provider display, a patient input device, a patient display, and software stored in the memory device, the software being readable and executable by the processor. Each of the patient interface device, patient display, provider interface device, and provider display is operatively connected to the processor. A provider account database is stored in the memory device, the provider account database storing a plurality of provider accounts corresponding to a plurality of providers, each provider having patients, the provider's patients being comprised in a plurality of patients corresponding to a plurality of patient accounts, each provider account comprising provider-side healthcare information of each of the corresponding provider's patients, the provider account having unique provider login credentials. A patient account database is stored in the memory device, the patient account database storing the plurality of patient accounts corresponding to the plurality of patients, each patient account comprising patient-side healthcare information of one of the patients, the patient account having unique patient login credentials. The software comprises instructions for the processor, in response to receiving provider login credentials entered into the provider input device which match the provider login credentials of the provider account, to display interactive provider dashboard views corresponding to the provider account on the provider display. The software further comprises instructions for the processor, in response to receiving patient login credentials entered into the patient input device which match the patient login credentials of one of the patient accounts, to display interactive patient app views corresponding to the patient account on the patient display. The provider dashboard views comprise a patient table view displaying patient data for each of the provider's patients, the patient data including a patient name, a patient diagnosis, a next step corresponding to the diagnosis having an agreed upon due date, an owner, and a patient risk level status, the owner representing a role responsible for the next step, and the risk level status representing that a due date has been missed, or otherwise representing a likelihood that the next step will not be completed by the agreed upon due date. The patient views display the patient's name, the patient diagnosis, the next step, and the risk level status.

### BRIEF DESCRIPTION OF THE DRAWINGS

Although the characteristic features of this disclosure will be particularly pointed out in the claims, the disclosed method and system, and how it may be made and used, may be better understood by referring to the following description taken in connection with the accompanying drawings forming a part hereof, wherein like reference numerals refer to like parts throughout the several views and in which:

FIG. 1 is a simplified block diagram of a healthcare management system according to this disclosure.

FIG. 2 is a view of a provider dashboard of the healthcare management system.

FIG. 3 is a view of a provider dashboard of the healthcare management system.

FIG. 4 is a view of a patient app user interface of the healthcare management system.

FIG. 5 is a view of a patient app user interface of the healthcare management system.

FIG. 6 is a view of a provider dashboard of the healthcare management system.

FIG. 7 is a view of a provider dashboard of the healthcare management system.

FIG. 8 is a view of a provider dashboard of the healthcare management system.

FIG. 9 is a view of a provider dashboard of the healthcare management system.

FIG. 10 is a view of a patient app user interface of the healthcare management system.

FIG. 11 is a view of a provider dashboard of the healthcare management system.

FIG. 12 is a view of a provider dashboard of the healthcare management system.

FIG. 13 is a view of a provider dashboard of the healthcare management system.

FIG. 14A is a view of a provider dashboard of the healthcare management system.

FIG. 14B is a view of a provider dashboard of the healthcare management system.

FIG. 14C is a view of a provider dashboard login screen.

FIG. 14D is a view of a provider dashboard of the healthcare management system.

FIG. 14E is a view of a provider dashboard of the healthcare management system.

FIG. 14F is a view of a provider dashboard of the healthcare management system.

FIG. 15 is a simplified graphical illustration of a method of use of the patient app.

FIG. 16 is a simplified graphical illustration of a method of use of the patient app.

FIG. 17 is a simplified graphical illustration of a method of use of the patient app.

FIG. 18 is a simplified graphical illustration of a method of use of the patient app.

FIG. 19 is a simplified graphical illustration of a method of use of the patient app.

FIG. 20 is a simplified graphical illustration of a method of use of the patient app.

FIG. 21 is a simplified graphical illustration of a method of use of the patient app.

FIG. 22 is a simplified graphical illustration of a method of use of the patient app.

FIG. 23 is a simplified graphical illustration of a method of use of the patient app.

FIG. 24 is a simplified graphical illustration of a method of use of the patient app.

FIG. 25 is a simplified graphical illustration of a method of use of the patient app.

FIG. 26 is a simplified graphical illustration of a method of use of the patient app.

FIG. 27 is a simplified graphical illustration of a method of use of the patient app.

FIG. 28 is a simplified graphical illustration of a method of use of the patient app.

FIG. 29 is a simplified graphical illustration of a method of use of the patient app.

FIG. 30 is a simplified graphical illustration of a method of use of the patient app.

FIG. 31 is a simplified graphical illustration of a method of use of the patient app.

FIG. 32 is a simplified graphical illustration of a method of use of the patient app.

FIG. 33 is a simplified graphical illustration of a method of use of the patient app.

FIG. 34 is a simplified graphical illustration of a method of use of the patient app.

FIG. 35 is a simplified graphical illustration of a method of use of the patient app.

FIG. 36 is a simplified graphical illustration of a method of use of the patient app.

FIG. 37 is a simplified graphical illustration of a method of use of the patient app.

FIG. 38 is a simplified graphical illustration of a method of use of the patient app.

FIG. 39 is a simplified graphical illustration of a method of use of the patient app.

FIG. 40 is a simplified graphical illustration of a method of use of the patient app.

A person of ordinary skill in the art will appreciate that elements of the figures above are illustrated for simplicity and clarity and are not necessarily drawn to scale. The dimensions of some elements in the figures may have been exaggerated relative to other elements to help to understand the present teachings. Furthermore, a particular order in which certain elements, parts, components, modules, steps, actions, events and/or processes are described or illustrated may not be required. A person of ordinary skill in the art will appreciate that, for simplicity and clarity of illustration, some commonly known and well-understood elements that are useful and/or necessary in a commercially feasible embodiment may not be depicted to provide a clear view of various embodiments per the present teachings.

### DETAILED DESCRIPTION

In the following description of various embodiments of the disclosed system and method, reference is made to the accompanying drawings, which form a part hereof, and in which various environments of the disclosed system and ways of practicing the disclosed method in those environments are illustrated by way of example. Other specific arrangements of systems, and environments, can be used, and structural modifications and functional modifications can be made without departing from the scope of the disclosed system and method.

Too often in healthcare settings, patients get “lost” in the system. For example, a patient struggles with a pending diagnosis and then never follows up to get the results of a biopsy. By the time the patient is seen again, often in an emergency room, they have lost treatment options and opportunities because their disease has advanced. The present disclosure provides a system for addressing this need.

As illustrated in the accompanying drawings and described herein, the present disclosure provides a computer-implemented healthcare management system and method. The system interfaces a patient-side application (“patient app”) with a provider dashboard to help facilitate collaboration and communication between patients and providers as well as promoting more active patient involvement in following up with next steps in their healthcare plan, rather than solely relying on provider task completion and provider-initiated follow-up. The system can be implemented in multiple healthcare settings of varying type and scale, where care may be provided by community health workers, low-income health providers, small medical practices, and/or large hospitals, clinics, or other healthcare organizations.

In one aspect, for each patient tracked by the system, the system displays in a provider's dashboard view a perceived level of risk that the patient will be “lost to follow-up”, which is to say, the level of risk that a next step in the patient's care plan will be completed untimely or not at all (referred to herein interchangeably as a “status,” “risk level,” or “risk level status”). A patient's risk level status is initially set by the provider team treating a patient and can be subsequently adjusted, either manually by the provider team or automatically by the system software. More particularly, the system software can detect the occurrence of an event that, as defined by the system programming, or as learned by system AI, indicates that the patient's care plan is not proceeding as expected, or otherwise increases the likelihood of the patient being lost. In response to such an event, the system can automatically raise the patient's risk level status. For example, in a preferred embodiment, the system automatically raises the patient's risk level status to the next higher level in response to detecting that an agreed upon due date for a next step has passed without the next step being logged in the system as completed, by provider input being entered into the provider dashboard and/or by patient input entered into the patient app. In embodiments, the system can likewise respond to a detected positive event, such as one or more next steps being completed early or on time, by automatically lowering a patient's risk level status, such as to the next lower level.

Such automated risk level status adjustment can be fully centralized, be fully decentralized, or occur in a blended centralized/decentralized manner. In an example embodiment of a system with fully centralized automated risk level status adjustment, a patient's risk level status is adjusted automatically whenever a system server or other centralized system computer or computer network detects a triggering event, such as by a clock signal indicating that the due date has passed without the system server having received a message, triggered by provider input via the dashboard or patient input via the patient app, and in response pushing out the risk status adjustment to provider devices, to the patient device of the subject patient, and to any other patient device to which the subject patient has granted access to the patient's profile.

In an example embodiment of a system with fully decentralized automated risk level status adjustment, system software running locally on a patient or provider device is programmed to adjust automatically a locally displayed risk level status upon locally detecting the triggering event, such as the passage of time without input from the provider or patient local user indicating next step completion, without the requirement of receiving any signal from the system server. More particularly, the locally running system software can be programmed to adjust the locally displayed risk level status only if the local device has not received next-step completion input from the local user (provider or patient). In still other embodiments, automated risk level status adjustment can occur in a blended centralized/decentralized manner. For example, if a due date for a patient-owned next step has past, despite the patient having entered timely input of next-step completion, the patient's locally displayed risk level status will nevertheless be raised automatically unless the local device has received confirmation from the system server that the next-step completion has been logged by the server. Still more strictly, the patient's locally displayed risk level status can be raised automatically unless the local device receives confirmation from the system server that the next-step completion has been logged by the server and transmitted to at least one provider device, and that the provider device has confirmed receipt of that server transmission. Such erring on the side of raising a locally displayed risk level status can serve to remind or alert the patient to a cause of the non-communication of the patient's next-step completion input, for example, the system server crashing or going offline; the patient device losing its internet connection; or that the patient has not logged in to his or her patient portal/account on the patient device since inputting the next-step completion (for example, having done so while using the app in an “offline mode” while out of range of a wi-fi or mobile telecommunications data signal, or using a computer or a different mobile device).

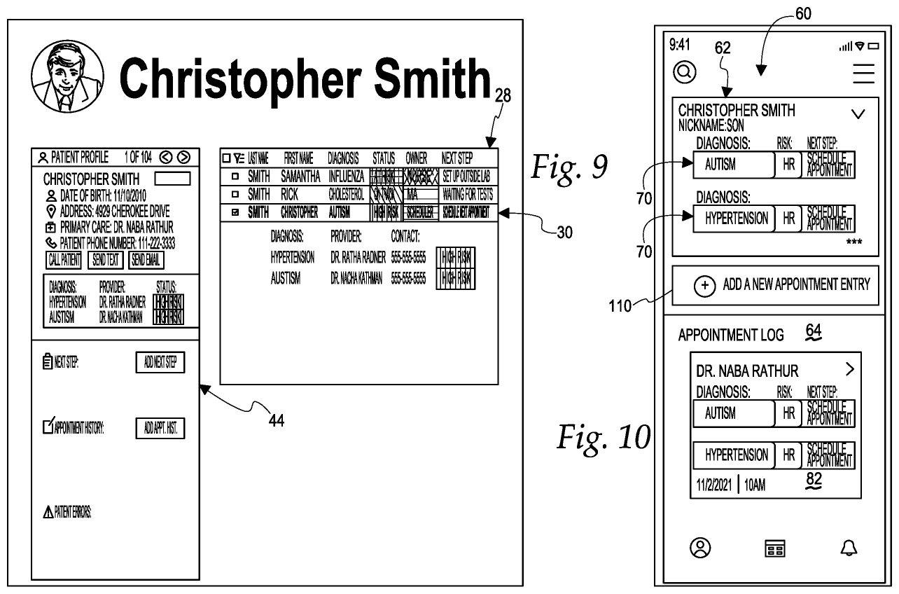

In the illustrated embodiment, the provider dashboard displays a table listing patients by name, diagnosis, risk level status, owner (role responsible for a next step), and next step. In this manner, the provider dashboard gives the provider a bird's-eye view of the risk levels of the provider's patients, so that the provider can use their work time wisely, being focused on the highest need and most urgent patients during their limited workday. Providers are also given the ability to calculate numbers and percentages of patients lost to follow-up, as a reflection of the overall quality of their (or their organization's) service to their patients, accounting not only for individual care interactions but also care plan management. In the dashboard view, providers can see the risk level status and next steps for their own patients and other providers' patients, facilitating proactive workload sharing among providers to reduce burn-out. The system can also track multiple diagnoses/conditions for the same patient, enabling a provider seen by the patient for one diagnosis to clearly see and communicate to the patient that the patient has a high-risk status related to a diagnosis for which another provider is treating the patient. For example, a patient has diabetes and breast cancer and follows up with the diabetes clinic but needs to be getting breast imaging. While interacting with the patient, the provider on the diabetes team will open the dashboard and notice an elevated risk status for the patient. The provider clicking on the risk status indicator will open an expanded view of all the patient's tracked diagnoses, risk statuses, and associated next steps, which will reveal that the elevated risk status is associated with a next step in that patient's breast cancer care plan. (An example of such an expanded view for a patient with a different pair of tracked diagnoses, autism and hypertension, is illustrated in FIG. 9 as a dropdown expansion of a patient row in a patient table view.) On seeing this, the provider will redirect the patient to the breast team.

Risk level status and next steps for the patient are also clearly displayed in the patient app, giving patients a window into how their medical team views their personal risk. In addition, the patient app displays each of a patient's multiple diagnoses simultaneously on a home page/screen, each diagnosis in an initially collapsed, single row of text cells showing the diagnosis, risk level status, and next step. This provides the patient a compact overview of all of the patient's next steps in a minimal amount of viewing space, reducing the likelihood that the details of any one diagnosis/care plan will crowd another diagnosis/care plan out the display area and/or distract the patient's attention, potentially causing the patient to miss a next step for another diagnosis.

A patient can also use the app to grant other patients viewing and/or interactive/editing access to the patient's account. One patient account can manage or be managed by one or more than one other patient account (such as one “lead” or “admin” patient managing the patient accounts of the lead patient's children and spouse, or multiple parent or guardian patients co-managing their child's patient account).

In another aspect, the system enables the provider and the patient each to view patient care plan information in their respective primary languages. For example, what a primarily English-speaking provider enters into the provider dashboard in English for a primarily Spanish-speaking patient will be automatically translated and displayed on the patient app in Spanish.

In another aspect, the system provides a safety net for patients by creating opportunities to reduce errors at multiple points in a patient's care plan. For example, the patient app prompts the patient to ask the provider key questions prior to the patient leaving a visit. For example, when a patient's appointment is scheduled on the system, the app can be triggered to launch automatically on the patient's smartphone/other device at the scheduled appointment time and to display prompts to assist a patient with self-advocacy during the visit. Advantageously, this facilitates confirmation that a provider and patient are on the same page. In addition, the app can be triggered to present automated care reminders to the patient at agreed upon timing, based on the patient's needs and not limited by the provider's availability to contact the patient.

In another aspect, the system ensures that tasks for each patient are clearly connected to the provider responsible for the task, by assigning an “Owner” to each “Next Step” in a patient's care plan. Advantageously, this avoids patients being confused about whom to contact, or providers duplicating or omitting the performance of tasks, as a patient's care team often includes multiple providers, such as doctors in different departments, or a nurse in one department and a scheduler in another. In another aspect, agreed upon due dates trigger triage status (including but not necessarily limited to automatically adjusting risk level statuses) and are automated for the provider and patient.

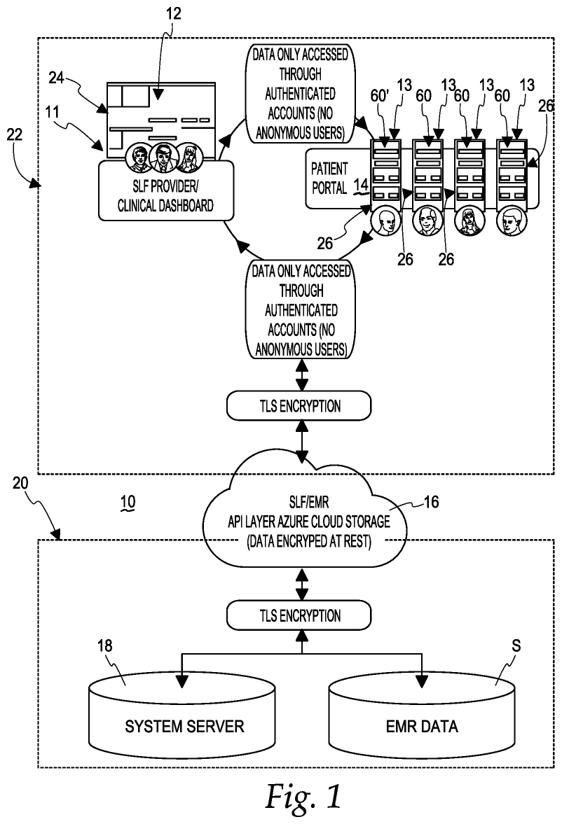

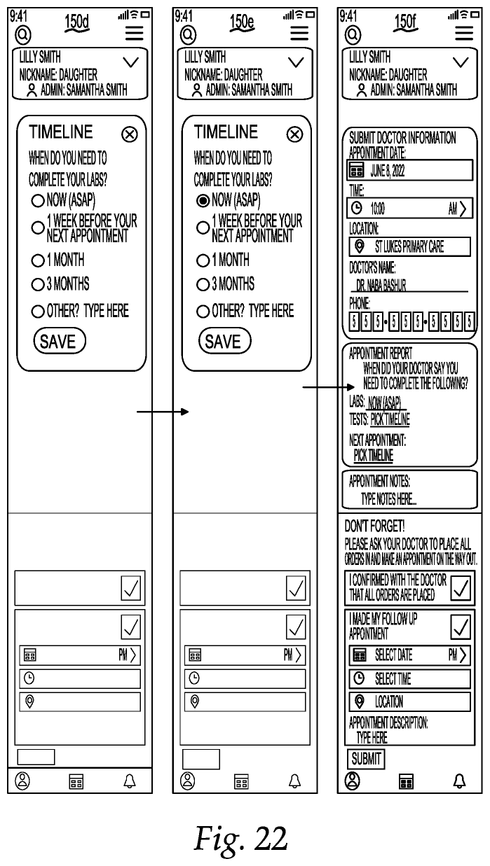

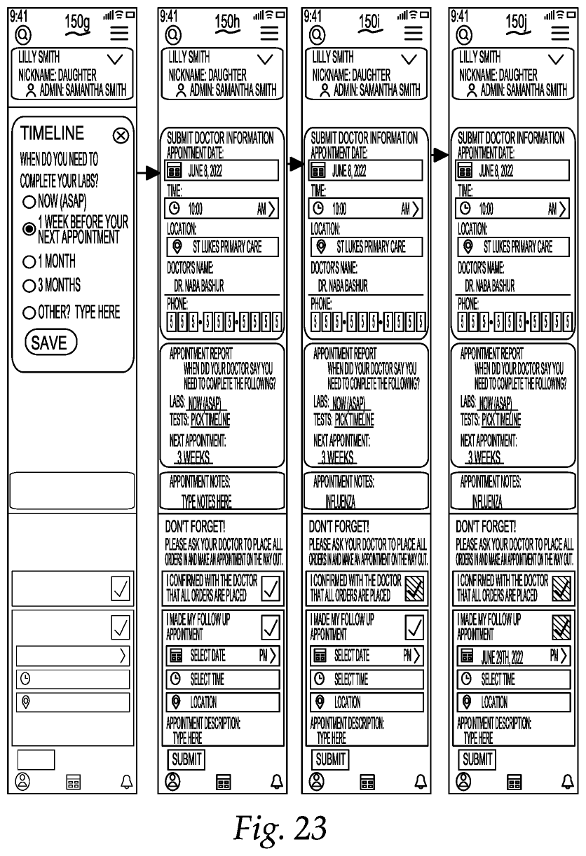

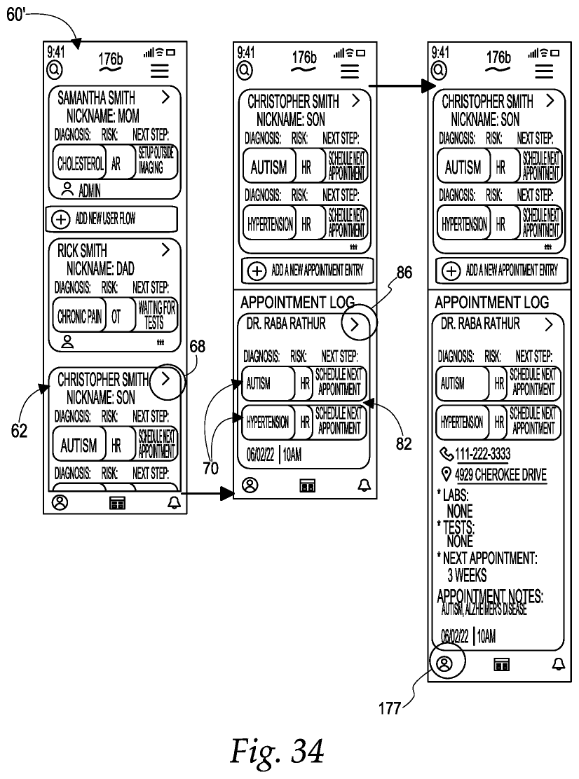

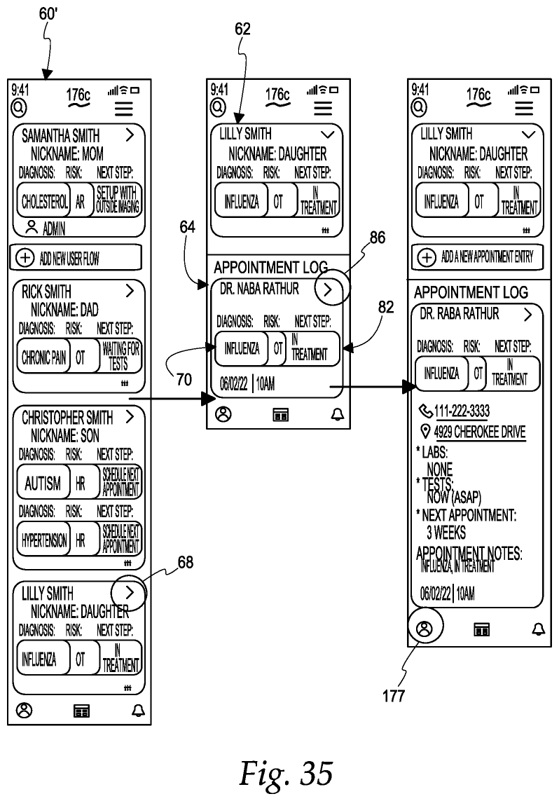

An embodiment of a healthcare management system according to the disclosure will now be described in greater detail, with reference to the diagrams and screenshots shown in FIGS. 1-40.

With reference to FIG. 1, the structure of a computer-implemented healthcare management system 10 is illustrated in a simplified block diagram. The system 10 includes at least one provider device 11 operative to run a provider application so as to display a provider/clinical dashboard 12 (also referred to as the “dashboard 12” or “provider dashboard 12,” said to include a navigable set of “provider views”) for interfacing with healthcare providers, a plurality of patient devices 13, each patient device 13 being operative to run a patient app so as to display a patient portal 14 (said to include a navigable set of “patient views”) for interfacing with healthcare patients, an encrypted cloud storage and application-program interface (API) layer 16 (“cloud storage layer 16” or “API layer 16”), and a system server 18. The system 10 is operative to interface (if needed) with an outside electronic medical records (EMR) storage S. The storage S may, for example, be part of another management system used by providers and/or their organization, such as a hospital, clinic, or medical practice group.

The system server 18 and the existing EMR storage S are operatively connected each other and to the cloud storage/API layer 16 via an external network, such as the internet or worldwide web. Collectively, a server side 20 of the system 10 is said to comprise the system server 18, the existing EMR storage S, and their secure connections to each other and to the cloud storage/API layer 16. This enables two-way secure transfer of patient health data between the cloud storage layer 16 and each of the system server 18 and the outside EMR storage S, the data being encrypted during transfer according to a suitable internet communications security protocol, such as TLS, and while at rest in the cloud storage layer 16. Similarly, a user side 22 of the system 10 is said to comprise the provider device 11, the patient devices 13, and secure connections between each of them and the cloud storage/API layer 16.

The provider device 11, which comprises a provider display 24, can be any suitable electronic device operative to run the provider application so as to display the dashboard 12 on the display 24 and to receive input to the dashboard 12 from the provider, such as a desktop or laptop computer (not shown) operatively connected to the display 24 and to a user input device such as a keyboard, mouse, or touch pad (not shown) and/or a touch screen comprised in the display 24. Typically, the provider device 11 has a large enough display 24 to display simultaneously the names, diagnoses, statuses, and next steps for a number of patients of the provider, such as twenty to thirty patients, in a format that is readily and comfortably readable by a provider having ordinary vision unaided by a magnification device, many times throughout a typical workday of the provider. Thus, the provider device 11 is typically either a computer, with a monitor or laptop screen as the display 24, or a tablet having a 7- to 13-inch screen as the display 24. However, the provider device 11 can also be a smartphone or similar device.

Likewise, the patient devices 13, which comprise patient displays 26, can be any suitable electronic device operative to run the patient app so as to display the patient portal 14 on the display 26 and to receive input to the patient portal 14 from the patient. Typically, each patient enrolled in the system 10 has at least one patient device 13 that is a smartphone or other portable device, the display 26 being a smartphone touch screen that also serves as the interface for patient input to patient portal 14. It is advantageous that the patient device 13 be portable and normally carried by the patient throughout a typical day, so that the patient device 13 can serve as a reliable mode of delivering real-time reminders or alerts to the patient wherever they may be, as well as displaying self-advocacy prompts to a patient during a patient's care visit to a provider location, according to another aspect of the system 10. Such reminders, alerts and/or prompts can be automatically set up and triggered by software of the system 10, which can include, without limitation, the provider application running on the provider device 11, the patient app running on the patient device 13, and/or a server application running on the system server 18. In addition, or alternatively, the patient may have a patient device 13 that is a desktop or laptop computer or a larger-format tablet that the patient may or may not typically carry to in-person care visits, but which the patient may use at home or elsewhere to access the patient portal 14.

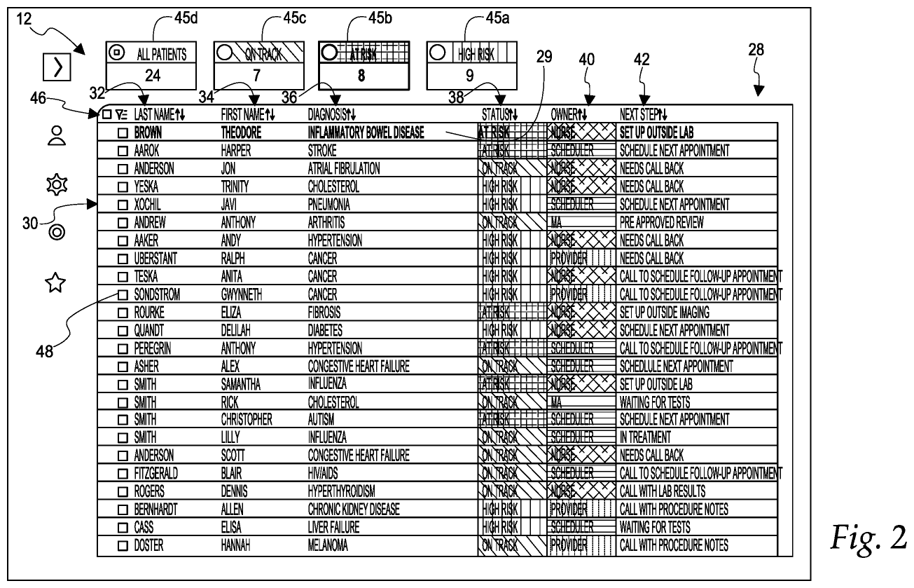

The tracking of next steps and risk level statuses for a group of patients of a healthcare provider will now be described with reference to various illustrative views or screenshots of the provider dashboard 12 and patient portal 14 shown in FIGS. 2-10. Turning to FIG. 2, a patient table 28 is displayed in the provider dashboard 12, which provides a high-level entry point for tracking the care plans of a plurality of patients. The patient table 28 has data values stored in a plurality of cells 29 of each a plurality of patient rows 30. More particularly, each patient row 30 comprises cells containing data corresponding to a single patient, including cells in last- and first-name columns 32, 34, a diagnosis column 36, a risk level status (or “status”) column 38, a next step owner (or Owner) column 40, and a next step column 42. The Owner column specifies not individuals but rather categories or types of Owners, which are illustrated, by way of non-limiting example, to include the Owner types Nurse, Scheduler, MA (Medical Assistant), and Provider. The Owner type “Provider” does not capture all “small-p” healthcare providers as that term is used herein, but rather only those Owners (all of whom are considered to be “small-p” healthcare providers for purposes of this disclosure) who are not nurses, schedulers, or medical assistants. For example, without limitation, a physician, radiologist, or clinical psychologist can be classified as a “capital-P” Provider type of Owner. Also, additional or different categories of Owner can be represented in the provider dashboard 12 without departing from the scope of this disclosure.

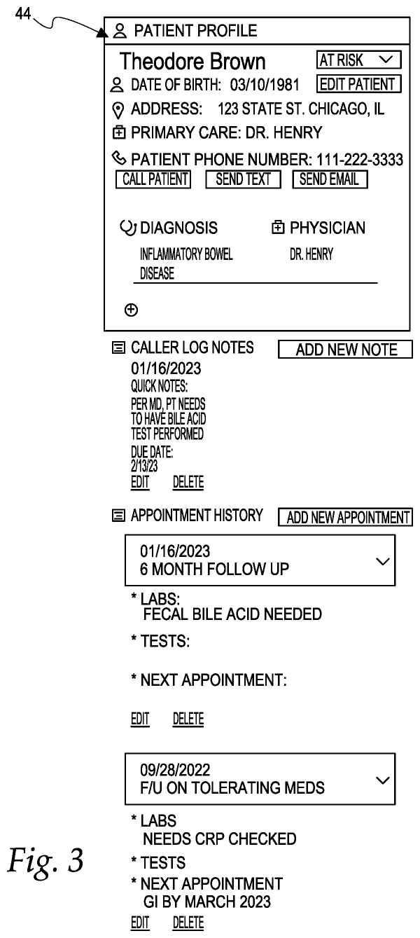

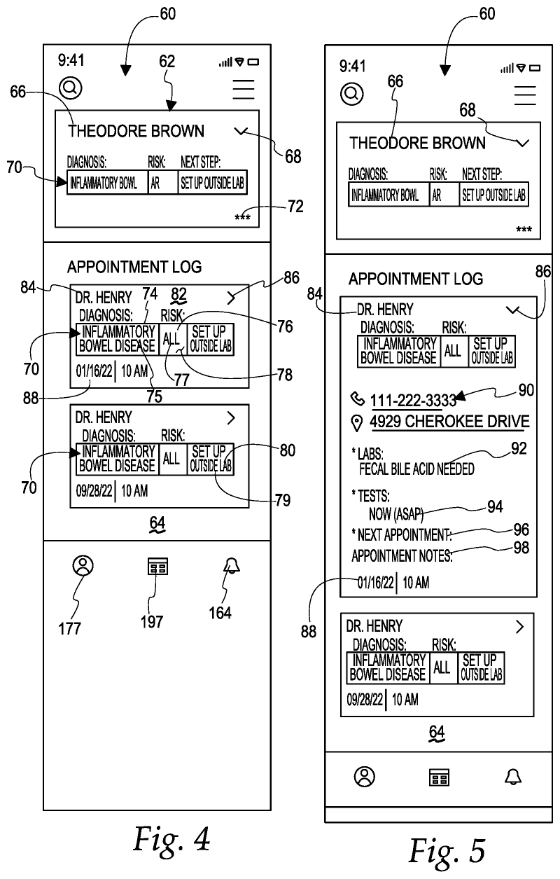

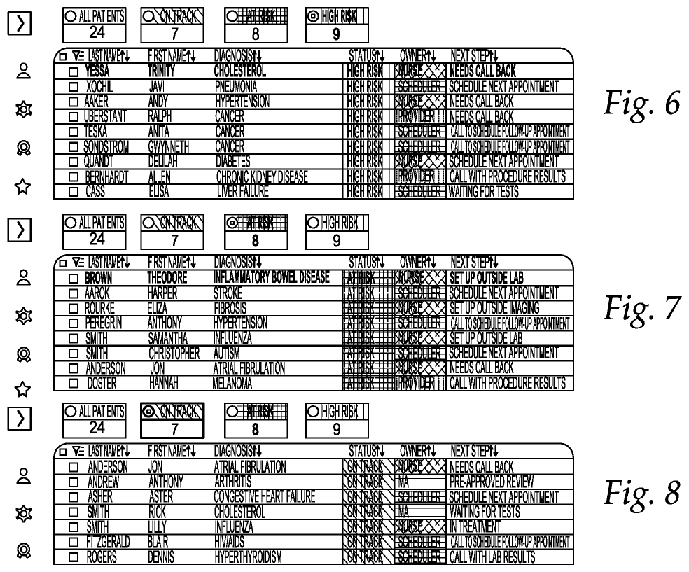

The provider dashboard 12 can also display patient profiles 44, as illustrated in FIGS. 3 and 4 for a hypothetical patient named Theodore Brown, the patient profile 44 including a display of the patient's name, risk level status, identification data such as a date of birth, and contact information. In addition, the patient profile 44 is illustrated to include a display of a diagnosing provider name and an appointment history section. The appointment history section is in turn adapted and configured to display an appointment date and an appointment description for each past appointment of the patient. A patient profile 44 can be displayed, for example, when a provider selects the corresponding patient row 30 of the table 28, such as by clicking anywhere in one of its cells 29, so that the row 30 is highlighted as seen in FIGS. 2 and 6-8. Although shown separately in FIGS. 3 and 4 for ease of illustration, the dashboard 12 is preferably able to display the patient profile 44 arranged or docked next to the patient table 28, as shown in FIGS. 6-8 for the hypothetical patients Trinity Yessa, Theodore Brown, and Jon Anderson, respectively. Illustrated in FIGS. 4 and 5, for context, is the patient portal 14 displayed for the same Theodore Brown, which he will see when he logs into his patient account through the patient app running on his own patient device 13.

The patient table 28 is sortable by the values in any of the six columns 32, 34, 36, 38, 40, 42, for example by a standard/commonly known user input method such as “clicking” or “tapping” a column header of the respective column in a header row 46. The table 28 is then sorted, for example, alphabetically by patient last name, first name, diagnosis, owner, or next step, or in descending or ascending order of risk level status, which is illustrated as having the values “High Risk” (highest risk level status, displayed with a red cell background), “At Risk” (intermediate risk level status, displayed with a yellow cell background), and “On Track” (lowest risk level status, displayed with a green cell background). It will be noted that background colors, such as “red,” “yellow,” “green” and others, may be represented symbolically in the accompanying drawing figures as different shades of grey and/or as different hatch-fill line patterns. Unless stated otherwise in this description, the same such shade or pattern shall be understood to represent the same color wherever it appears in the drawings.

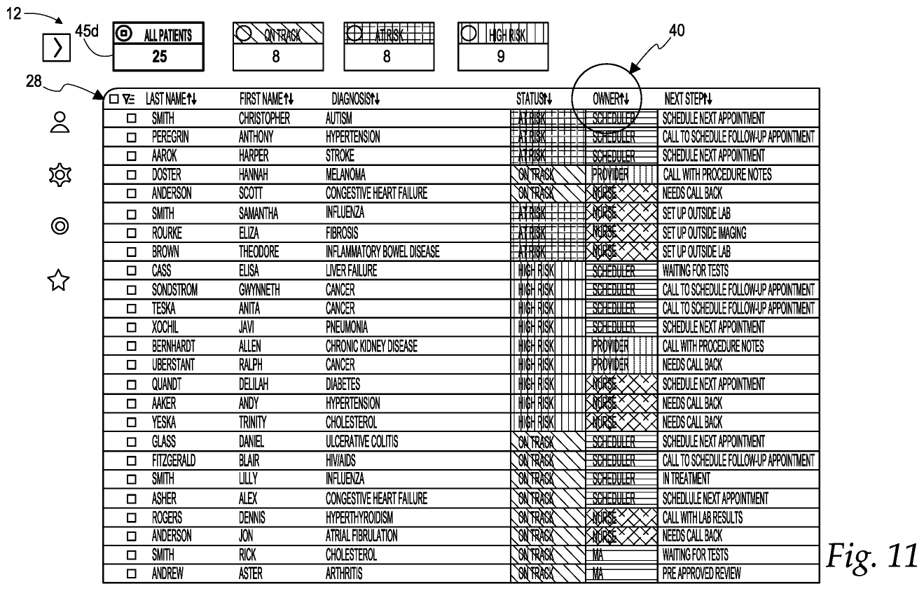

The table 28 being sortable by the data in any of its columns facilitates certain batch operations, as in the example of sending next-step notifications for a batch of patients to a scheduler who is the next step owner 40, described further below with reference to FIGS. 11-14B. In addition, displayed above the patient table 28 are a set of similarly color-coded High Risk, At Risk, and On Track filter controls 45a, 45b, 45c, one of which can be selected by toggling a corresponding radio button to display only the patient rows 30 having the corresponding risk level status 38, as shown in FIGS. 6-8, respectively, as well as an All Patients filter control 45d, which removes all filters so that all patients are displayed in the table 28 regardless of their risk level status 38. At all times, a count of all patients remains displayed in the filter control 45d, and a count of patients in the respective risk level status category remains displayed in each of the filter controls 45a-c. In this manner, all the patients remain represented, at least at a high level, in all views of the patient table 28, rather than lower risk patients falling completely out of view when the provider focuses on higher risk patients. The filtered views of the table 28 shown in FIGS. 6-8 are likewise sortable by column in the same manner as the unfiltered view of the table 28 shown in FIG. 2.

In the illustrated embodiment, a patient with multiple diagnoses is nonetheless listed in only one patient row 30 of the table 28, with just one of the patient's diagnoses being displayed automatically. However, the provider user can expand a given patient's information in the dashboard view to see data for additional diagnoses, such as by clicking anywhere in the corresponding patient row 30, by clicking more particularly in the cell 29 of the risk status 38, by checking a selection box 48, or by selecting a drop-down arrow (not shown). The resulting drop-down view is illustrated in FIG. 9 for the patient row 30 of a hypothetical patient named Christopher Smith, where information for both the patient's diagnoses (hypertension and autism) is displayed.

The single diagnosis 36 displayed in the table 28 for a patient can, for example, default to that for which the patient has the highest present risk level status 38. In other embodiments, the single diagnosis 36 displayed by default can be that which was diagnosed most recently or that for which the patient has the earliest approaching due date for a corresponding next step 42.

In an embodiment, each individual provider has a unique individual provider account in the system 10, and for a given patient, preference will automatically be assigned to displaying a diagnosis of that patient, if any, that is managed by the provider who is currently logged into the provider dashboard 12. For example, an individual physician who diagnosed or has seen or treated the patient for a particular diagnosis 36 will see that diagnosis displayed in the table 28 by default.

In another embodiment, regardless of which single diagnosis is displayed in a collapsed view of a patient row, when a patient has multiple actively tracked diagnoses, a visual indicator (not shown) appears in to indicate that the patient corresponding to the patient row has additional diagnoses. For example, an expansion control may be displayed in or adjacent the patient row, for example as a “three-dots”/“overflow” symbol or a dropdown arrow, which can be activated to expand the patient row to view information for each diagnosis. In still other embodiments not shown, a patient's multiple diagnoses are automatically displayed in a single diagnosis cell, for example written out as a list on separate lines or separated by commas. Regardless of which diagnosis is displayed by default, the risk level status 38 displayed for the patient can, in embodiments, automatically be the highest risk level status 38 for any of the patient's diagnoses. In this manner, a provider caring for any of the patient's diagnoses will automatically be alerted to an elevated risk status 38 for the patient and have the opportunity to assist by, for example, directing the patient to the owner 40 of the next step 42 having the elevated risk level, which the viewing provider can identify by opening the patient's drop-down view.

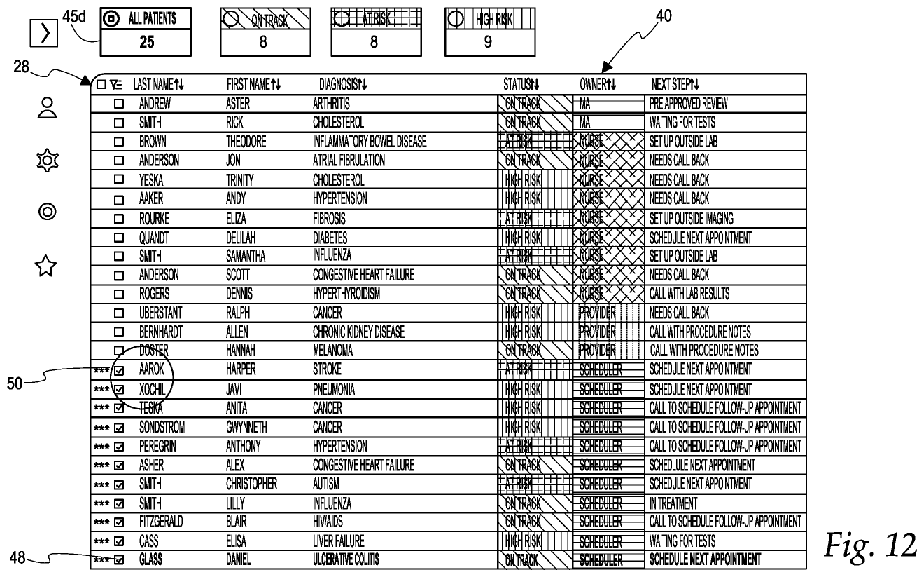

Batch user operations facilitated by the system 10 are now described in detail, with reference to an example illustrated in FIGS. 11-14B. In the illustrated example, a provider user, for example a head nurse, clicks the column title “Owner” in the header row 46 in the “All Patients” view of the patient table 28, as shown in FIG. 11. This results in the table 28 being sorted by next step owner 40 as shown in FIG. 12, where it is further illustrated that a provider user has selected all the patient rows 30 having “Scheduler” as the next step owner 40. Conveniently, these patient rows now being consecutive in the sorted table 28 makes them easier to find, particularly with color coding by type of owner, as well as making it easier to perform the selection, either by mouse-clicking each individual selection box 48, or executing a multi-selection input, for example one of the standard multi-selection inputs “shift+click,” “ctrl+click,” “ctrl+shift+click,” “click-and-drag” (to draw a selection area), or other suitable input, which may be standard, user-customized, or defined for use in the provider dashboard application software component of the system 10.

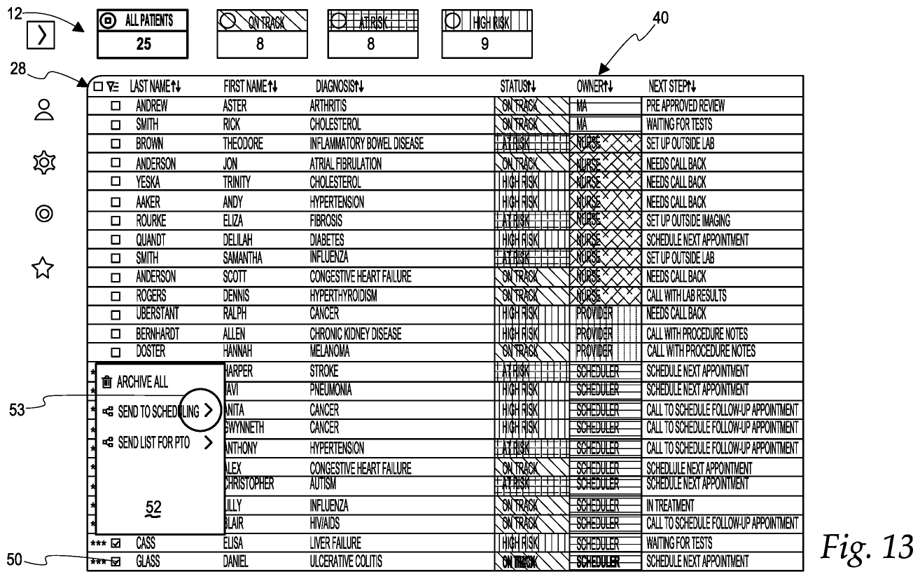

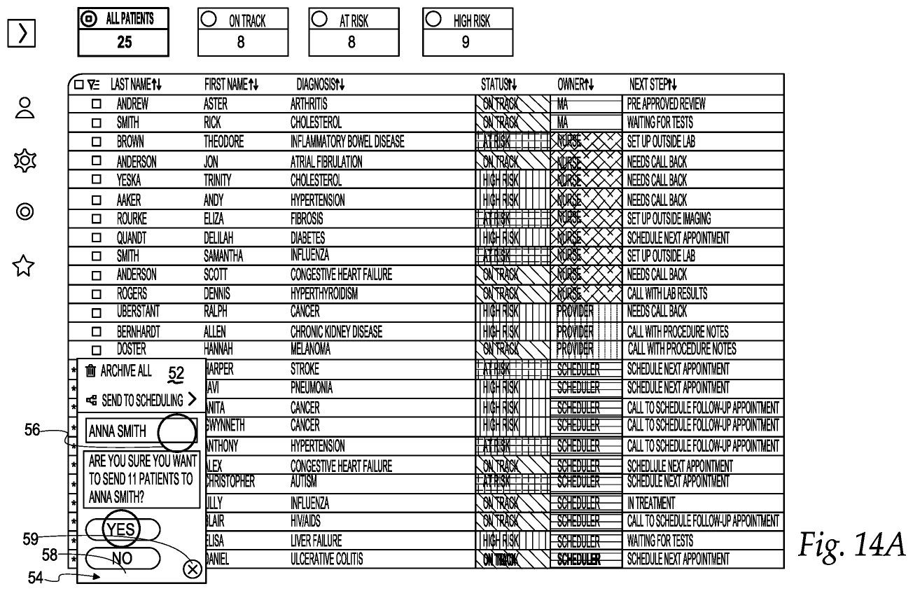

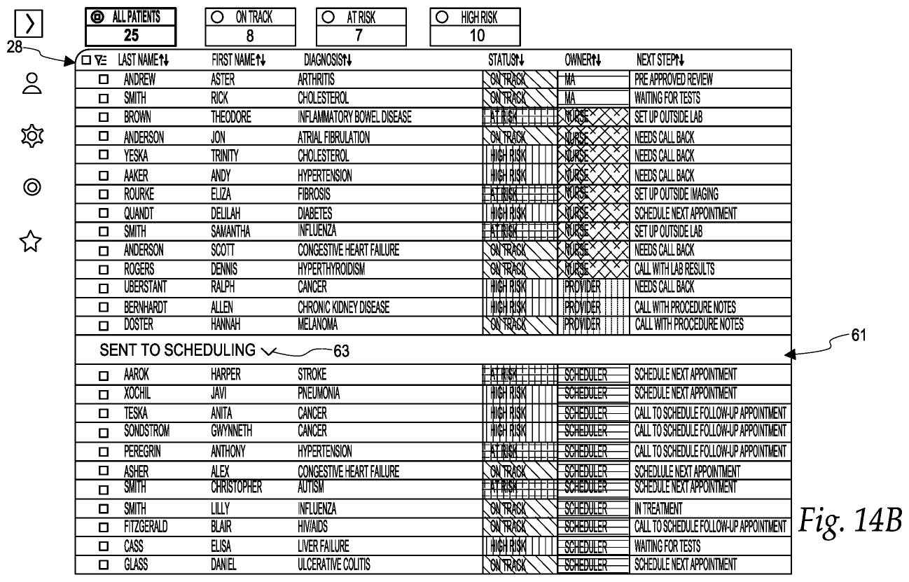

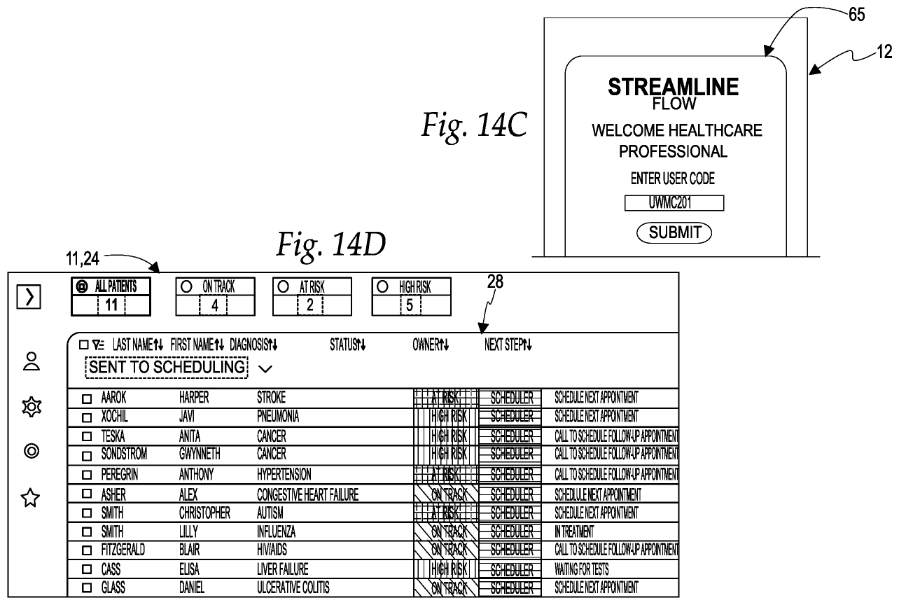

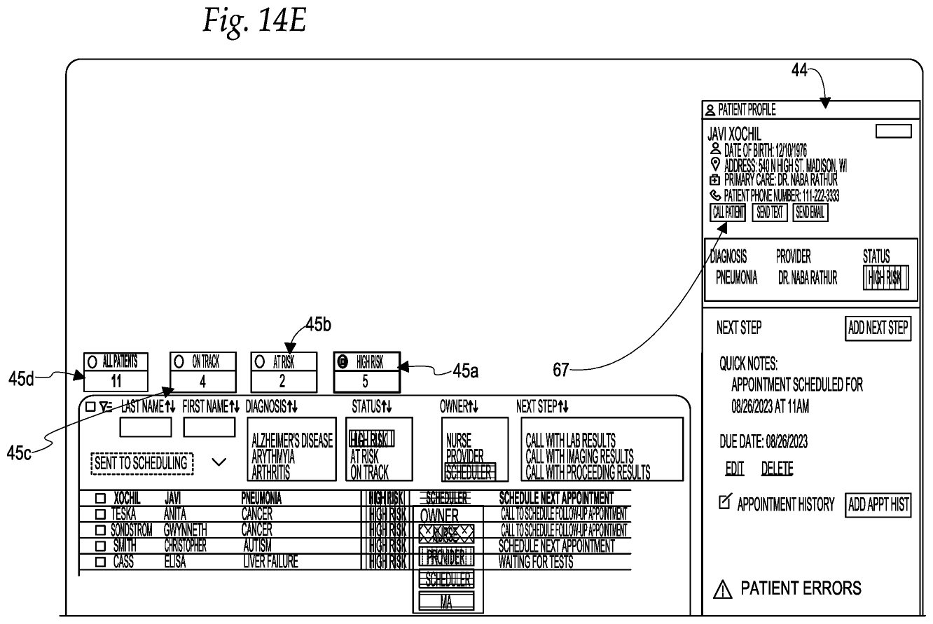

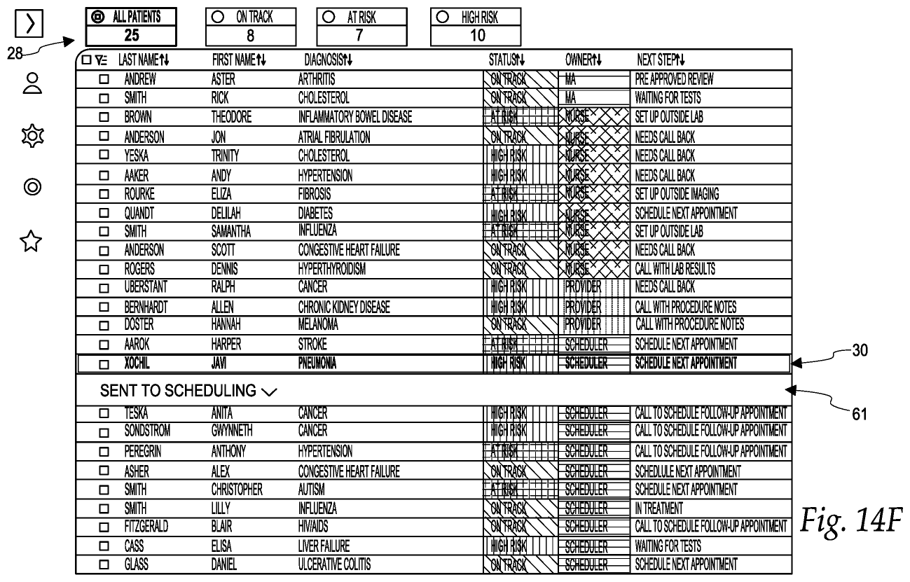

As illustrated in FIG. 13, the provider then activates the three dots control 50 to open a batch operations menu 52 of including “Archive all,” “Send to scheduling,” and “Send list for PTO.” Next, the provider clicks a dropdown control 53 on the “Send to scheduling” line to expand a recipient selection and confirmation menu 54. Within the menu 54, the provider can activate another dropdown control 56 to expand a dropdown list of recipient choices and choose a recipient from the dropdown list, as shown in FIG. 14A, followed by activating a “Yes” or “No” button in a confirmation control panel 58 to either initiate sending eleven patients to the selected Scheduler (shown as Anna Smith) (“Yes”) or cancel the sending operation (“No”). In case the “No” button is activated, the the confirmation menu 54 typically will remain “open” (displayed in the foreground) awaiting the provider user's choice whether to select a different recipient using the drop-down control 56, select a different batch operation by closing the confirmation menu 54 by activating a Close control 59 (appearing as an X) to return to the batch operation options, or to close the batch operations menu 52 altogether by clicking or tapping outside the batch operations menu 52 to return to the owner-sorted view of the patient table 28, for example as shown in FIG. 11 with the selection of “Scheduler” rows 30 cleared, or as shown in FIG. 12 with that selection still active.

In case the “Yes” button is activated, the dashboard 12 will display a confirmation that the selected patients have been sent to scheduling and/or more specifically to the selected Scheduler. In the illustrated embodiment, such confirmation is provided by partitioning the patients in the patient table 28 who were sent to scheduling from those who were not, optionally preceded by a pop-up notification indicating the name of the Scheduler to whom the patients were sent, followed by displaying the partitioned patient table 28 when the pop-up notification is closed. More particularly, as shown in FIG. 14B, a horizontal bar 61 containing the text “Sent to scheduling” and a dropdown control 63 is inserted into the patient table 28 with the patients that were sent to scheduling displayed below the bar 61 and those who were not sent to scheduling displayed above it. Activating the dropdown control 63 allows the provider dashboard user to expand or collapse/hide the group of patients that were sent to scheduling. It will be understood that such batch sending saves a provider user, such as a head nurse, time during their limited workday by consolidating the repetitive task of sending a number of patients to scheduling into a single batch task requiring a greatly reduced number of user input control activations.

According to another aspect of the system 10, system software is programmed with instructions for the provider dashboard 12 to automatically notify a provider assigned to a patient when a scheduler has not initiated contact or been able to contact a patient within a preset bounce-back time limit after the patient was sent to scheduling. For example, the provider may be prompted (not shown) to select this preset bounce-back time limit whenever sending a given patient or batch of patients to scheduling, or the preset bounce-back time limit may be set according to a system-wide rule. This saves the head nurse the additional time of having to set him or herself a reminder to follow up on whether the patient was reached. More generally, such automated bounce-back of patients can be implemented any time one provider sends a patient or group of patients to another provider, regardless of whether the receiving provider belongs to the same Owner type as the sending provider. In embodiments, the sending provider can be prompted to choose whether to schedule such an automated bounce-back, as well as to select triggering conditions, including but not limited to a bounce-back time limit. More particularly, whether scheduling a bounce-back is required or at the option of the sending provider can depend on the circumstances of the transmission; for example, the provider dashboard application may require the sending provider to schedule a bounce-back whenever sending a patient to a Scheduler, but a bounce-back may be optional for certain other types of transmissions.

Turning now to FIGS. 14C-F, there is illustrated the receiving Scheduler Anna Smith's receipt and handling of the list of patients sent as illustrated in FIGS. 11-14B and described above. Shown in FIG. 14C is Anna's login portal 65 (which may be representative of that of any provider having a provider account in the system 10) as displayed by the provider dashboard 12, where Anna enters and submits her user code to log in to the dashboard 12. The system server 18 and/or the cloud storage/API layer 16 receives and authenticates Anna's user code, upon confirmation of which authentication Anna's provider device 11 is instructed to display Anna's view of the dashboard 12 on its display 24, which is shown in FIG. 14D to include a patient table 28 showing only those patients that have been sent to scheduling. Turning to FIG. 14E, Anna then filters her view of the patient table 28 to display only High Risk patients by activating the High Risk filter control 45a, facilitating her handling scheduling of High Risk patients first. (Similarly, she may then proceed to handle At Risk patients by activating the filter control 45b and then performing the following described steps to schedule each of those patients.) Next, Anna selects the patient row of the High Risk patient Javi Xochil to bring up his patient profile 44, which includes a call control 67 displayed next to the patient's phone number. Anna proceeds to call Javi Xochil, either by manually dialing the phone number or by activating the call control 67, schedules his appointment over the phone, and edits a Next Step section of the Patient Profile to indicate that an appointment has been scheduled and the scheduled date and time. Then, Anna selects the Owner cell in Javi Xochil's patient row to open an Owner selection box and selects Nurse from a panel of selectable owner types displayed in the box, thereby changing the designated Owner for Javi Xochil from Scheduler to Nurse. The system server 18 and/or the API layer 16 receives notification of this change from Anna's provider device 11 and, in turn, updates all providers' views of the dashboard 12 to reflect the change. The head nurse's view of the patient table 28 is updated as shown in FIG. 14F, by moving Javi Xochil's patient row 30 up from below the “Sent to scheduling” partition bar 61 to above it, as well as reflecting the Owner change to Nurse.

The patient app according to the illustrated embodiment of a healthcare management system and method will now be described in detail, with reference to FIGS. 4, 5, 10, and 15-40. Turning to FIGS. 4 and 5, two illustrative views are shown of the patient portal 14 of the hypothetical patient Theodore Brown. System 10 software includes instructions for the patient device to display a patient user interface 60 when a patient has logged into the patient portal 14 via the patient app running on the patient mobile device 13. The user interface 60 includes a patient header panel 62 and an appointment log panel 64. The patient header panel 62 displays a patient name 66, a drop-down control 68, a diagnosis status bar 70 (if the patient is being treated for a diagnosis, or multiple bars 70 for more than one diagnosis), and a three-dots control 72, the diagnosis status bar 70 comprising a diagnosis cell 74 containing diagnosis text 75, a risk cell 76 containing a risk level status 77 in a color-coded background 78 corresponding to a risk level status for the corresponding next step, and a next step cell 79 containing next step text 80 for the next step

The appointment log panel 64 can display one or a plurality of appointment boxes 82, each appointment box 82 including provider name text 84, a dropdown control 86, date and time information 88 for a corresponding appointment, and a copy of each diagnosis status bar 70 corresponding to each diagnosis related to the corresponding appointment, thus redundantly reinforcing the communication of the corresponding risk level status 77 to the patient, which corresponds to, and normally equates to, the risk level status 38 displayed for that patient in the dashboard patient table view 28. As previously described, any discrepancy between the dashboard and patient portal risk level statuses 38, 77 may be due to disruption of automated communications normally occurring between or among the patient device 13 running the patient app, the API layer/cloud storage 16, the system server 18, and/or the provider device 11 running the provider software of the system 10, where the discrepancy, if any, resulting from such a disruption can depend on the rules governing automated risk level status adjustment according to embodiment of the system 10 or a method of its use. As shown in FIG. 4, the patient user interface 60 is shown with each appointment box 82 collapsed, while the appointment box 82 for the appointment dated Jan. 16, 2022 is shown in FIG. 5 as expanded by the patient user activating the dropdown control 86 (for example by tapping the screen). The expanded view of the appointment box 82 additionally includes provider contact information 90, a Labs field 92 for notes regarding any completed or needed labs, a Tests field 94 for notes regarding any completed or needed tests, a Next Appointment field 96 for notes regarding a next appointment scheduled or to be scheduled, and an Appointment Notes field 98 for any other notes. The fields 92, 94, 96 may be editable by the patient or automatically populated with data entered by the provider via the provider dashboard 12, as directed by the API layer/cloud storage 16 and/or by the system server 18 upon their receipt of the provider-entered data.

In FIG. 10, a patient user interface 60 for the hypothetical patient Christopher Smith is shown, illustrating multiple diagnosis status bars 70 being displayed in the patient header panel 62 (one for each of Christopher Smith's diagnoses of autism and hypertension) as well as in an appointment box 82 for a Nov. 2, 2021 appointment relating to both diagnoses. In addition, the user interface 60 for Christopher Smith is illustrated as including an add appointment control 110 that is included for a patient having profile editing access. Patient user input activating the add appointment control 110 displays an additional, blank fillable appointment box (not shown) in the appointment log panel 64 and prompts the patient to manually enter, select from dropdown lists, or toggle options to be automatically inserted, as appropriate, provider name text, a copy the diagnosis bar 70 of each diagnosis related to the appointment, if any, a date and time, and any appropriate notes in an expanded view corresponding to that of the illustrated appointment box 82.

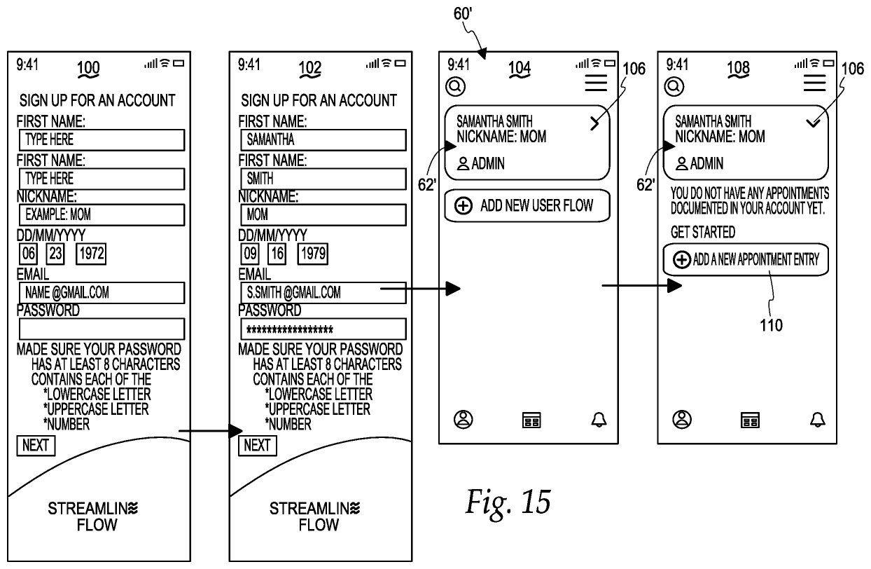

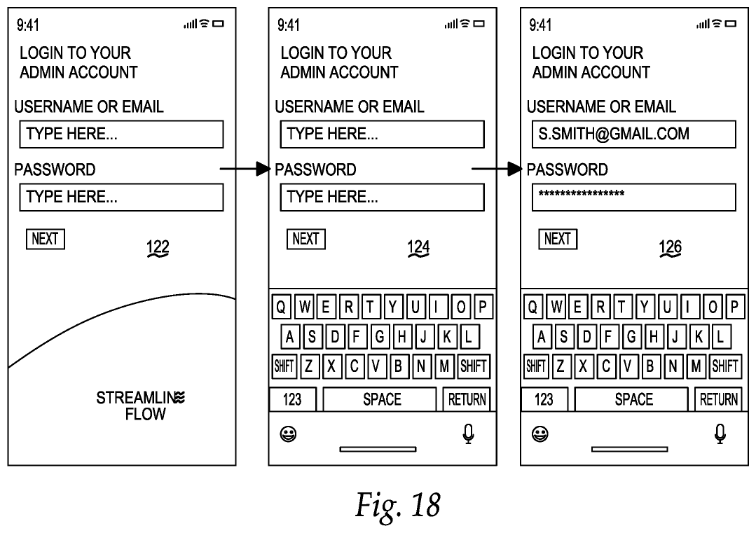

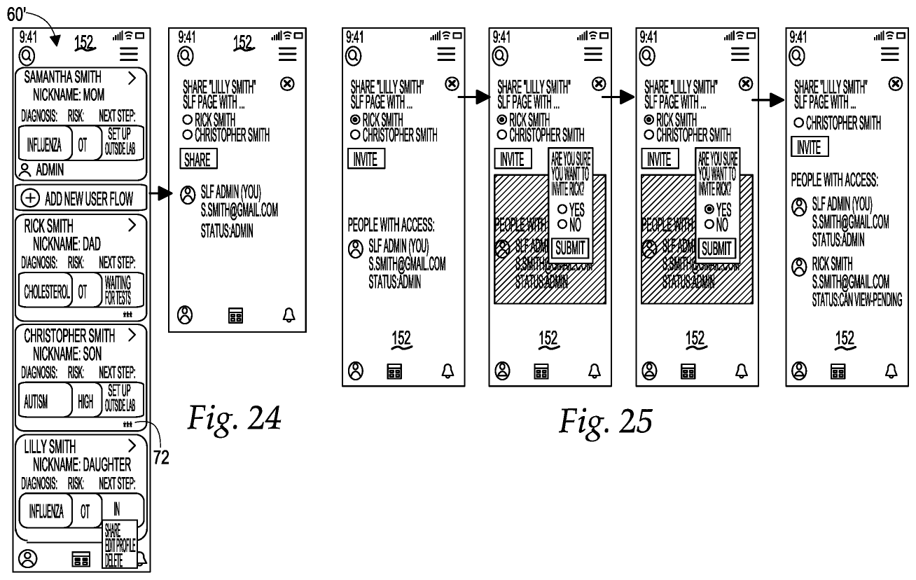

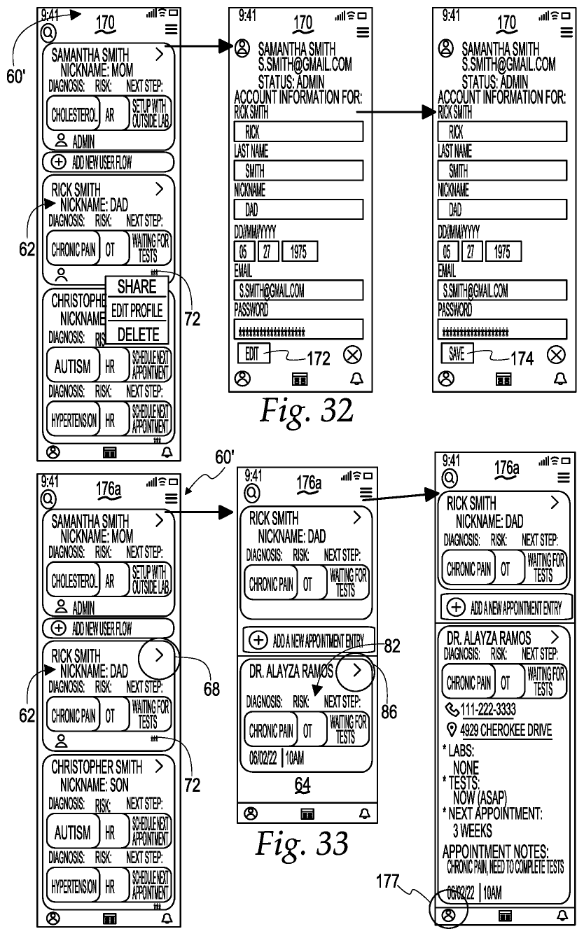

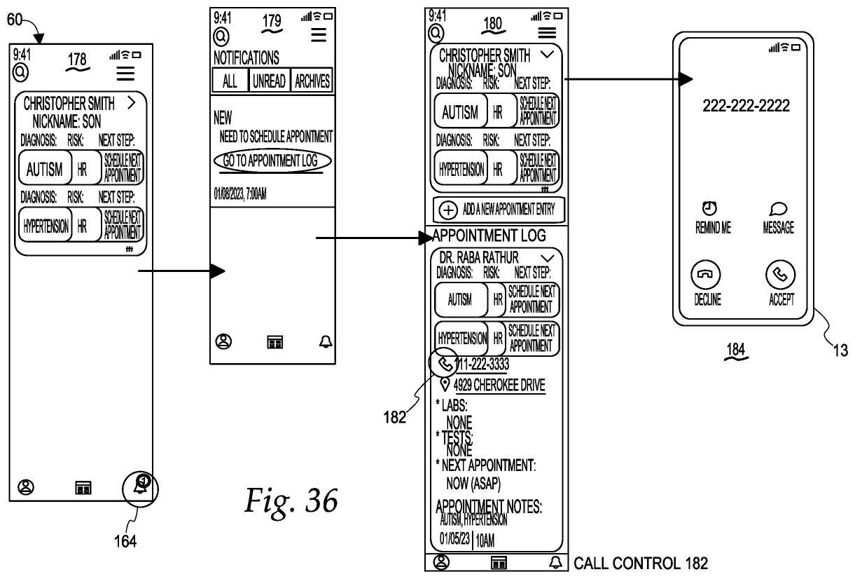

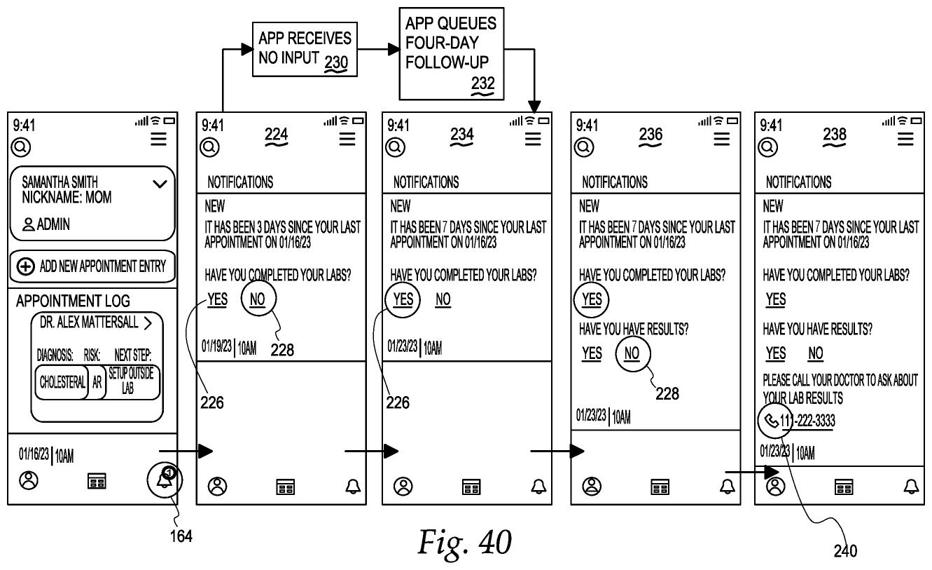

Procedures to sign up for (create) a patient account in the patient portal 14 and/or to define relationships and roles/permissions among existing patient accounts are illustrated in the simplified flow diagrams of FIGS. 15-40, showing screenshots of a patient app, in some of which the name “Streamline Flow” appears in plain text or as a logo, for illustrative purposes only, as a non-limiting example of the patient app and/or the system 10 as a whole being associated with a name or mark. More particularly, illustrated in FIGS. 15, 16, and 17 is method by which a hypothetical patient Samantha Smith creates and logs in to her new account and adds a new appointment entry. Illustrated in FIGS. 18-23 is a method by which Samantha Smith (Nickname: Mom) logs into an admin account (also referred to herein as a “lead patient” account) and adds a new patient user profile Lilly Smith (Nickname: Daughter). Illustrated in FIGS. 24-30 is a method by which Samantha Smith invites another user Rick Smith (Nickname: Dad) to view the new profile for Lilly Smith, which viewing access is sent to Lilly Smith for approval, approved by Lilly Smith, and granted. Illustrated in FIGS. 31-32 is a method by which Samantha Smith can edit sign-up information for users managed by her admin account. Illustrated in FIGS. 33-35 is a method by which Samantha Smith can access and interact with the patient app user interface for each patient user managed by her admin account. Illustrated in FIGS. 36-39 is a method by which Christopher Smith is notified of an appointment, Samantha Smith connects a calendar to her admin account, and Samantha Smith adds Christopher Smith's appointment to her calendar. Illustrated in FIG. 40 is a method by which the system provides automated reminders to Samantha Smith, prompts for her input, and takes further action in response to receiving her prompted input selections.

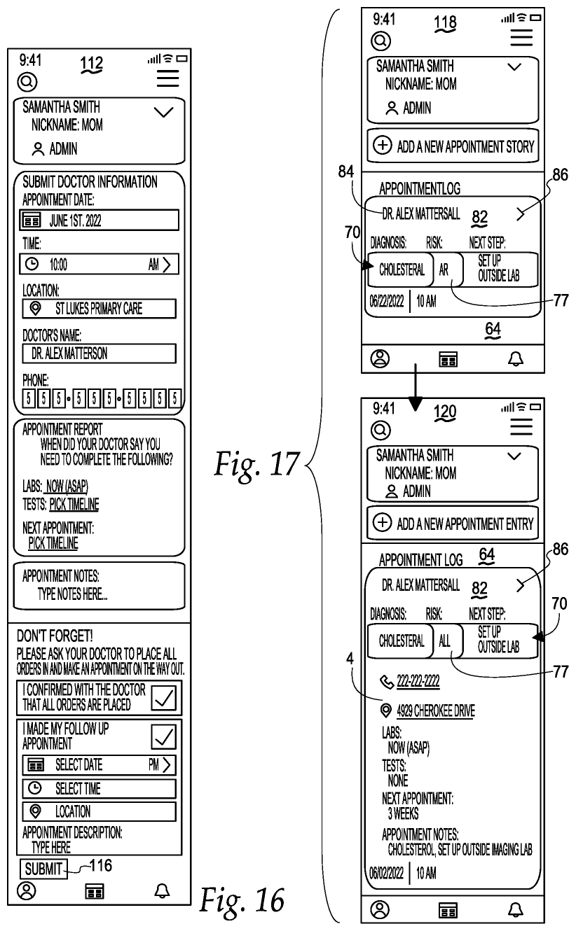

Turning to FIGS. 15, 16, and 17, a method of creating a new account and adding a new appointment entry is illustrated for the hypothetical patient Samantha Smith. In a in a step 100 shown in FIG. 15, Samantha Smith has installed and opened the patient app on her patient device 13 and is prompted by the app to sign up for an admin patient account by entering information in the displayed fields, which she enters in a step 102 and clicks a “Next” button. In a step 104, in response to Samantha's input, the app displays a new admin patient user interface 60′ for Samantha's newly created admin patient account. Input is received from Samantha, who taps a dropdown control 106 in an admin patient header panel 62′, and the app responds in a step 108 by displaying an Add a New Appointment Entry control 110. In response to Samantha tapping the Add control 110, the app displays an appointment information form in a step 112, which Samantha fills in and then clicks a Submit control 116, as illustrated in FIG. 16. In a step 118 illustrated in FIG. 17, in response to the activation of the Submit control 116, the patient app adds an appointment log panel 64 to Samantha's admin patient user interface 60′, analogous to the appointment log panel 64 as previously described for a general patient app user.

When the appointment entered by Samantha has taken place (which may have already happened when Samantha entered the information, that is, Samantha was recording a past appointment), the appointment log panel 64 will contain an appointment box 82 for the added appointment. In the illustrated example, the appointment resulted in the creation of a next step for Samantha's diagnosis of “Cholesterol.” Therefore, a diagnosis status bar 70 is displayed in the appointment box 82 for Samantha's diagnosis of “Cholesterol” related to the added appointment. The diagnosis bar 70 includes the risk level status 77 associated with Samantha's diagnosis, which the app has obtained automatically by querying the API layer/cloud storage 16 for the risk level status 38 assigned to the matching diagnosis 36 displayed in and stored in association with the provider dashboard patient table 28. In a step 120, also shown in FIG. 17, the patient app responds to Samantha tapping the dropdown control 86 by expanding the appointment box 82 to show the appointment information as received from Samantha in the step 112.

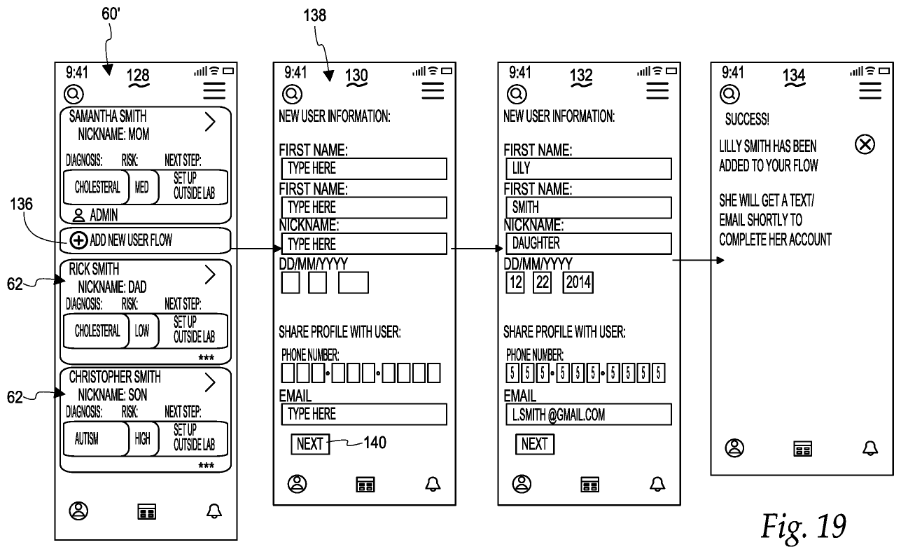

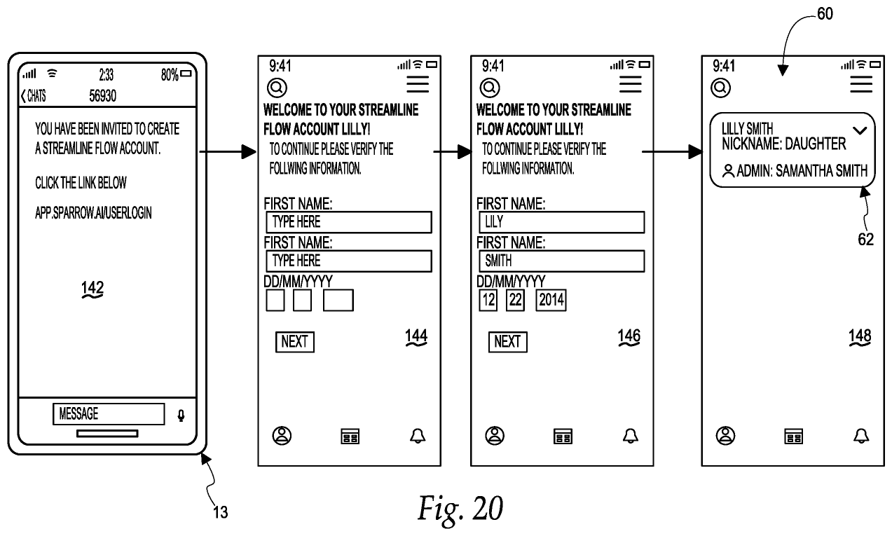

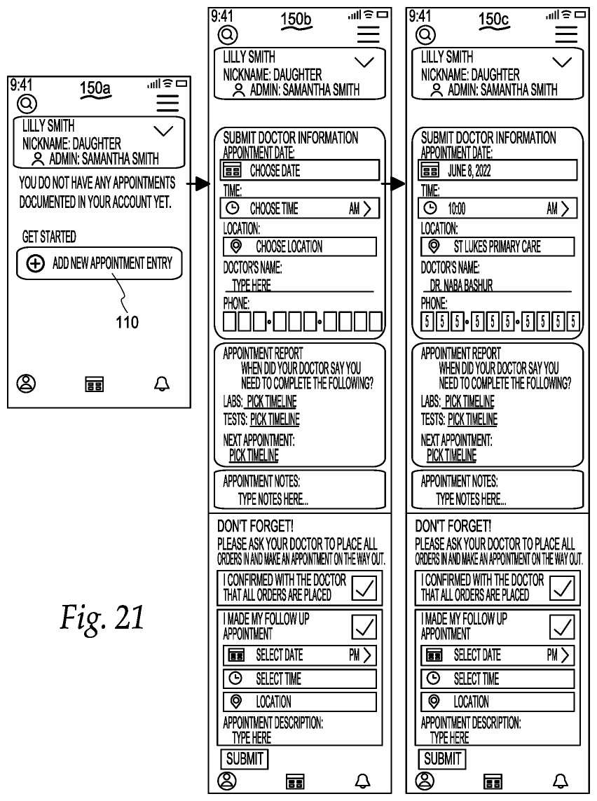

Turning to FIGS. 18-23, methods of adding and managing admin patient access to other patient accounts are illustrated. Shown in FIG. 18 are login steps 122, 124, and 126 of the patient app displaying a login interface, displaying a keyboard input interface in response to Samantha Smith tapping a login field, and receiving Samantha's login submission in response to Samantha tapping a Next control, respectively. Shown in FIG. 19 are steps 128, 130, 132, and 134. In the step 128, the app displays Samantha's admin patient user interface 60′ in response to receiving Samantha's login submission, the user interface 60′ including an Add New User Flow control 136, as well as patient header panels 62 for the previously added new (patient) users Rick Smith (Nickname: Dad) and Christopher Smith (Nickname: Son), by a process analogous to that shown in steps 130-134 for Lilly Smith (Nickname: Daughter). In response to Samantha tapping the Add control 136, the app displays a new user information input form 138 in the step 130. In the step 132, the patient app receives Samantha's input into the form 138 and her activation of a Next control 140, and in a step 134 the patient app displays a success message indicating that Lilly Smith has been added to Samantha's “flow.” In further response, the app directs the patient device 13 to submit a request to the API layer 16 to invite Lilly Smith to create a patient account, as illustrated in the steps 142, 144, 146, 148 shown in FIG. 20. In the step 142, the API layer 16 (and/or the system server 18) causes a text message including an account creation URL to be sent to Lilly's phone number as previously submitted by Samantha. In response to Lilly visiting the link, Lilly's patient device 13 (such as her smartphone, if she has tapped the link in the text message) displays a welcome message prompting her to enter her full name and birthdate in the step 144, and receives Lilly's input and submission in a step 146, followed by displaying Lilly Smith's newly created patient user interface 60 in the step 148. In FIGS. 21-23 are illustrated further steps 150a-j of an appointment being added to Lilly's patient account, as initiated by user input activating the Add control 110 previously described.

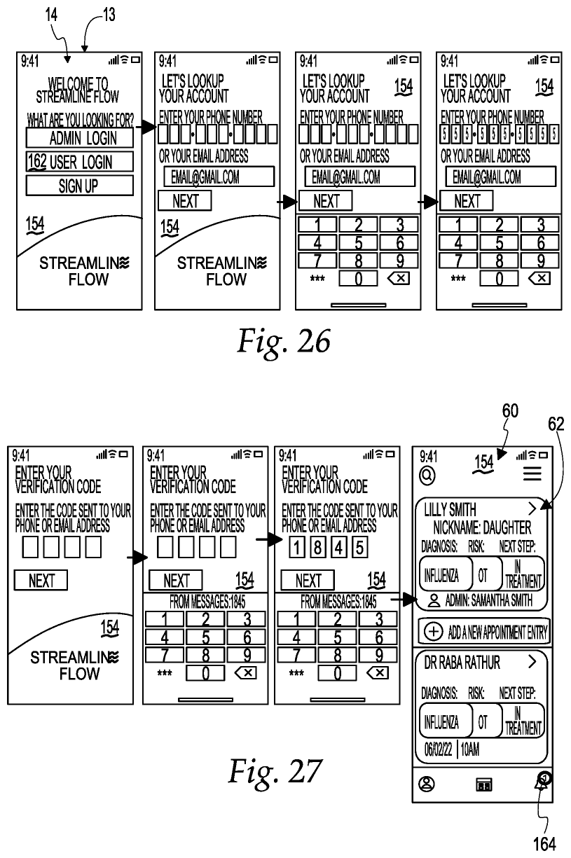

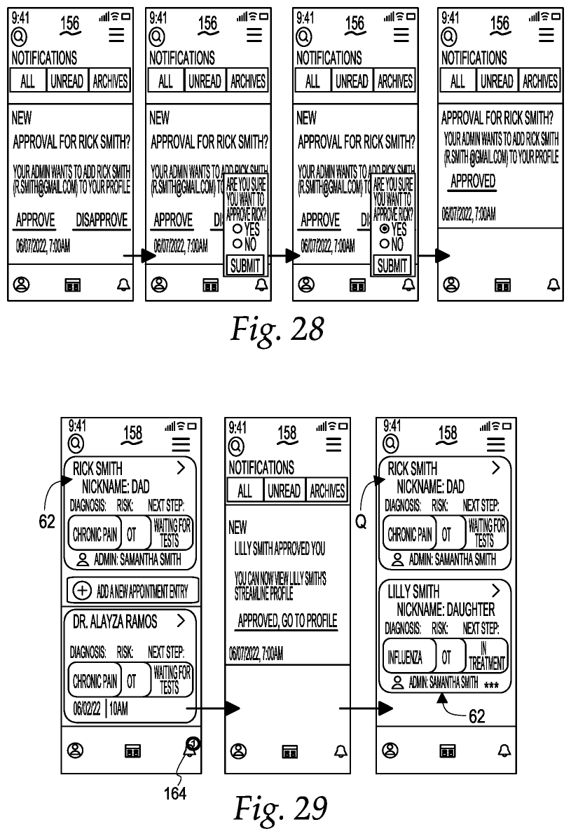

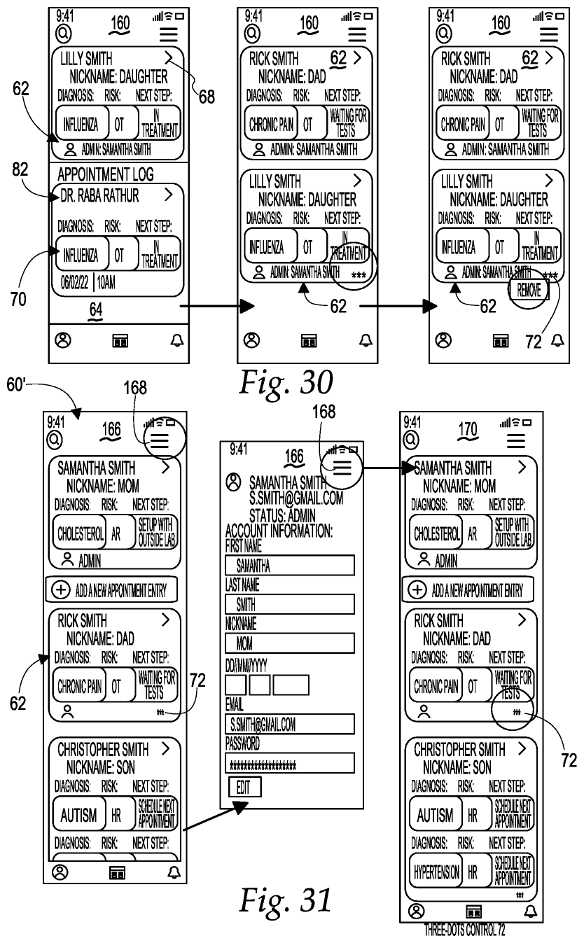

Turning to FIGS. 24-30, steps 152 (FIGS. 24-25), 154 (FIGS. 26-27), 156 (FIG. 28), 158 (FIG. 29), and 160 (FIG. 30) are illustrated, each having an illustrative sequence of substeps represented by screens connected by arrows to indicate a sequence in which they are displayed according to an embodiment. In the step 152, Samantha initiates sharing Lilly's profile with Rick by tapping to activate the three dots control 72 in Lilly's profile, selecting “Share,” as shown in the first screen of FIG. 24, and following prompts. In the step 154, Lilly logs into her account so that she can receive and approve the notification that Rick has been invited to view her profile. First Lilly opens the patient app on her patient device 13 and logs into the patient portal 14 by tapping a user login control 162 and responding to prompts to look up her account using her contact information, as shown in the four screens of FIG. 26, and then to log in to her account using a verification code, and in response to Lilly's verification code input, the app displays Lilly's user interface 60, where over a notification control 164 (shown as a “notification bell”) there is displayed a “1” indicating one new notification. In a step 156 in response to Lilly activating the notifications control 164, the app displays the new notification that the Admin wants to add Rick Smith, prompts Lilly for and receives Lilly's approval of the share operation, as illustrated in FIG. 28. In response to Lilly's approval submission, the app directs Lilly's patient device 13 to transmit an approval message to the API layer 16 (and/or to the server 18 via the API layer 16), which intern relays the approval message to Rick's user account. In response, in a step 158 shown in FIG. 29, the patient app running on Rick's patient device 13 displays a “1” on the notification control 164 of Rick's user interface 60, displays the notification and a go-to profile link in response to Rick activating his notification control 164, and displays Lilly's patient header panel 62 in response to Rick activating the link. In a step 160 shown in FIG. 30, in response to Rick activating the dropdown control 68 in Lilly's patient header panel 62, the app displays Lilly's appointment log panel 64 including an expandable appointment box 82 for Lilly's appointment that was added in steps 150a-j, but does not display an add appointment control 110, reflecting the fact that Rick Smith has only viewing and not admin access to Lilly's patient account. In addition, when Rick taps the three dots control 72 of Lilly's patient header panel 62, the only option presented to Rick is to “Remove” Lilly's profile.

Turning to FIGS. 31-32, profile editing functions in the patient app are illustrated for Samantha Smith's admin patient account. In a step 166, illustrated in the first two screenshots of FIG. 31, a form to edit Samantha Smith's own account information is displayed and toggled off by Samantha's successive activations of a hamburger menu control 168 (three horizontal lines). In a step 170 shown in the third screenshot of FIG. 31 and continued in FIG. 32, the patient app responds to Samantha's activation of the three-dots control 72 of Rick's patient header panel 62 by displaying options to “Share,” “Edit Profile,” or “Delete,” responds to Samantha's selection to “Edit Profile” by displaying a form containing Rick's Account information, responds to Samantha activating an “Edit” control 172 by unlocking the form fields for editing and displaying a “Save” control 174, and finally responds to Samantha's activation of the Save control 174 by returning Samantha to the home screen of her admin patient user interface 60′, as shown in the first screenshot of FIG. 33.

Turning to FIGS. 33-35, a step 176a, 176b, 176c of the patient header panel 62 being expanded to show the patient's appointment log panel 64, and then expanding an appointment box 82 to show appointment details, in response to Samantha's activation of the patient header panel dropdown control 68 and appointment box dropdown control 86, for each of the user profiles that she manages, of Rick Smith, Christopher Smith, and Lilly Smith, respectively, and then to return to a home screen of her admin patient account user interface 60′ by activating a home control 177. This illustrates Samantha's ability to review appointment information for each patient managed under her admin patient account via her admin patient user interface 60′.

Turning to FIGS. 36-39, there is illustrated a process by which the system 10 reminds a patient of the need to schedule an appointment and then facilitates the scheduling and calendaring of the appointment. Thus, as shown in the screenshots of FIG. 36, the patient app displays an indication of a notification in the home screen of Christopher Smith's patient app user interface 60 in a step 178, responds to Christopher's activation of the notification control 164 by displaying in a step 179 a notification that Christopher needs to schedule an appointment, including a link to go to Christopher's appointment log, responds to Christopher's activation of the link in a step 180 by displaying an expanded appointment box 82 containing a note “NOW (ASAP)” under the header “Next Appointment:” and a provider phone number with a call control 182, and responds to Christopher's activation of the call control 182 in a step 184 by causing Christopher's patient device 13 (in this case a smartphone) to direct a phone call to the provider phone number.

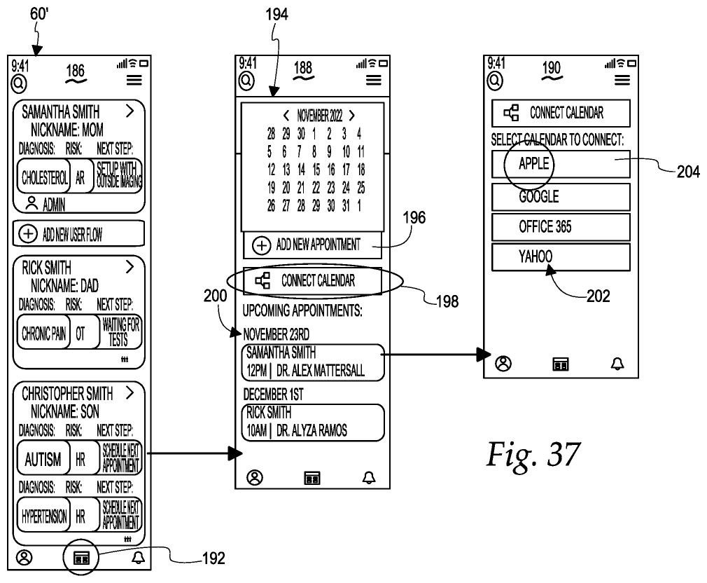

In FIG. 37 there are illustrated the steps 186, 188, 190 for Samantha to connect a calendar to her admin patient account. In the step 186 of the first screenshot, the app receives Samantha's input activating a calendar control 192 in her admin patient user interface 60′, and responds in a step 188 as shown in the second screenshot by displaying a calendar panel 194 including a monthly calendar image for November 2022 with dots indicating appointments on November 23^{rd} and December 1^{st}, an add appointment control 196, a connect calendar control 198, and an upcoming appointments list 200, in which the November 23^{rd} appointment for Samantha Smith is displayed in a block having a background color-coded to indicate that it is her own (admin patient) appointment and the December 1^{st} appointment for Rick Smith is displayed in a separate block having a background color-coded differently to indicate that it is an appointment of a patient user whose account is managed under her admin patient account, both blocks being of different colors than that of an overall background of the calendar panel 194. In response to Samantha's activation of the connect calendar control 198, the app displays in a step 190 a connect calendar menu 202 including selection controls 204 corresponding to choices of external calendars to connect. In response to Samantha activating one of the controls 204, the app will cause to be displayed an appropriate interface for Samantha to authenticate read-write access by the app to the external calendar.

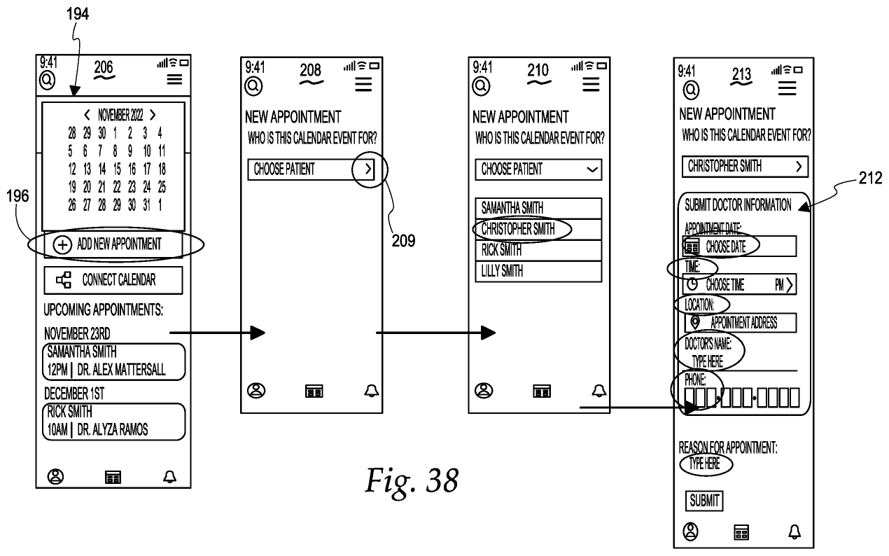

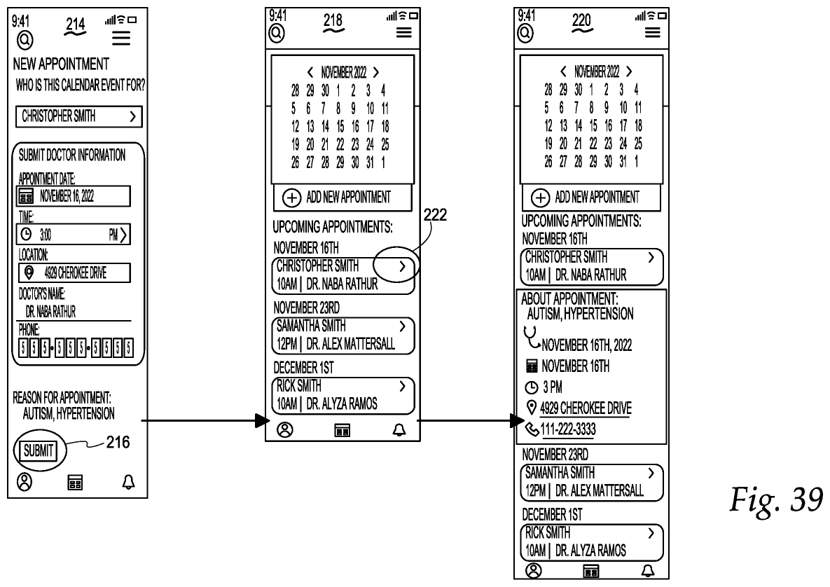

Turning to FIG. 38, in response Samantha's authentication of the external calendar, in a step 206, the app displays in the calendar panel 194 an indication that a calendar is connected, in place of the connect calendar control 198 of the previous view of the calendar panel 194. The app then responds to Samantha's activation of the add appointment control 196 by prompting Samantha to choose a patient for the calendar event in a step 208, responding to Samantha's activation of a choose patient dropdown 209 by displaying a list of patients in a step 210, and responding to Samantha's selection of Christopher Smith from the list by displaying a fillable appointment form 212 in a step 213. Continued in FIG. 39, a first screenshot illustrates a step 214 of the app receiving Samantha's inputs to the form 212 and activation of a form submission control 216. The app responds in a step 218 by again displaying the calendar panel 194, now with the newly added appointment for Christopher Smith on November 16^{th} added to the upcoming appointments list 200, in a block color-coded as an appointment for a managed patient user. In a step 220, the app responds to Samantha's activation of a dropdown control 222 for the November 16^{th} appointment by displaying the appointment details just submitted by Samantha under the associated block.

Turning to FIG. 40, there is illustrated an example of a patient self-advocacy function of the patient app according to the illustrated embodiment of the system 10. In a first screenshot of FIG. 40, there is displayed in Samantha Smith's patient app user interface 60 an indication of one notification, over the notification control 164. In a step 224 of the second screenshot, the app responds to Samantha Smith's activation of the notification control 164 by displaying a reminder notification that it has been three days since Samantha Smith's appointment on Jan. 16, 2023 and prompting Samantha Smith to indicate whether she has completed her labs, by tapping a yes control 226 or a no control 228. In a step 230, the patient app receives Samantha Smith's selection of the no control 228, triggering a step 232, in which the system software responds to the No input via the patient app by queueing a follow-up notification reminder to be sent to Samantha in four days. The follow-up reminder queueing step 232 can comprise scheduling the notification locally in the memory of a primary patient device 13 (such as Samantha Smith's smartphone), to be activated automatically at the scheduled time as measured by an internal clock of the patient device 13, whether or not the patient device 13 is at that time in communication with the API layer/cloud storage 16, and whether or not Samantha Smith is at that time logged into his account on the patient device 13. In addition or alternatively, the reminder queueing step 232 can comprise the app directing the patient device 13 to send a signal directing the API layer 16 to schedule the notification in the cloud storage 16 and/or in a memory of the system server 18, to be delivered automatically at the scheduled time, as measured by a clock of the API layer 16 and/or of the system server 18, to any patient device 13 that is then in communication with the API layer 16, on which Samantha Smith is at that time logged into the patient portal 14. Four days later, in a step 234 illustrated in shown in the third screenshot, the app presents a follow-up notification that is has been seven days since the appointment, again prompting Samantha to indicate whether she has completed her labs. In a step 236 illustrated in the fourth screen, the app responds to Samantha Smith's selection of the yes input 226 to the follow-up inquiry regarding lab completion on the previous screen by prompting Samantha to indicate whether she has test results. Upon receiving Samantha's selection of the No input 228, The app responds in a step 238 shown on the fifth screen, by displaying a prompt to call the doctor for next steps, including a call control 240 next to the doctor's phone number, in response to the activation of which the patient app would then cause the patient device 13 to direct a call to the displayed number, as previously illustrated for Christopher Smith in FIG. 36.

The preceding description of the disclosure has been presented for purposes of illustration and description and is not intended to be exhaustive or to limit the disclosure to the precise form disclosed. The description was selected to best explain the principles of the present teachings and the practical application of these principles to enable others skilled in the art to best utilize the disclosure in various embodiments and various modifications as are suited to the particular use contemplated. It should be recognized that the words “a” or “an” are intended to include both the singular and the plural. Conversely, any reference to plural elements shall, where appropriate, include the singular.

It is intended that the scope of the disclosure not be limited by the specification but be defined by the claim(s) set forth below. In addition, although narrow claims may be presented below, it should be recognized that the scope of this disclosure is much broader than presented by the claim(s). It is intended that broader claims will be submitted in one or more applications that claim the benefit of priority from this application. Insofar as the description above and the accompanying drawings disclose additional subject matter that is not within the scope of the claim or claims below, the additional disclosures are not dedicated to the public and the right to file one or more applications to claim such additional disclosures is reserved.

What is claimed is:

1. A computer-implemented healthcare management system comprising:
a processor;
a computer readable memory device operatively connected to the processor;
software stored in the memory device, the software being readable and executable by the processor;
a patient account database stored in the memory device, the patient account database storing a plurality of patient accounts corresponding to a plurality of patients;
a provider account database stored in the memory device, the provider account database storing a plurality of provider accounts corresponding to a plurality of providers;
each of the plurality of providers having patients, the provider's patients being comprised in the plurality of patients corresponding to the patient accounts, each provider account comprising provider-side healthcare information of each of the corresponding provider's patients, the provider account having unique provider login credentials;
each patient account comprising patient-side healthcare information of one of the patients, the patient account having unique patient login credentials;
a provider input device;
a provider display;
a patient input device;
a patient display;
each of the patient interface device, patient display, provider interface device, and provider display being operatively connected to the processor;
the software comprising instructions for the processor, in response to receiving provider login credentials entered into the provider input device which match the provider login credentials of the provider account, to display interactive provider dashboard views corresponding to the provider account on the provider display;
the software comprising instructions for the processor, in response to receiving patient login credentials entered into the patient input device which match the patient login credentials of one of the patient accounts, to display interactive patient app views corresponding to the patient account on the patient display;
the provider dashboard views comprising a patient table view displaying patient data for each of the provider's patients, the patient data including a patient name, a patient diagnosis, a next step corresponding to the diagnosis having an agreed upon due date, an owner, and a patient risk level status, the owner representing a role responsible for the next step, and the risk level status representing a likelihood that the next step will not be completed by the agreed upon due date;
the patient views displaying the patient's name, the patient diagnosis, the next step, and the risk level status.

2. The computer-implemented healthcare management system of claim 1, further comprising
the provider views further including a patient profile view for each of the provider's patients, the patient profile view displaying the patient name, the risk level status, patient identification data, patient contact information, a diagnosing provider name, and an appointment history section, the appointment history section being adapted and configured to display an appointment date and an appointment description for past appointments of the patient;
the patient views further displaying an appointment log panel, the appointment log panel being adapted and configured to display a plurality of displayed appointment boxes, each appointment box corresponding to an appointment of the patient.

3. The computer-implemented healthcare management system of claim 2, further comprising at least one appointment box displayed in the appointment log panel, each displayed appointment box having a collapsed view and an expanded view, the collapsed view displaying a provider name, appointment date and time information, an appointment diagnosis, an appointment diagnosis next step, and an appointment diagnosis risk level status, the at least one appointment box including an appointment box displaying the dashboard patient table view diagnosis, next step, and risk level status.

4. The computer-implemented healthcare management system of claim 1, the patient risk level status comprising a risk level selected from a ranked plurality of risk levels, the software further comprising instructions for the processor to receive input from at least one of the patient interface device and the provider interface device indicating the completion of the next step, and, in response to the passage of the agreed upon date without the next step completion input being received, to elevate the patient risk level status to the next higher risk level.

5. The computer-implemented healthcare management system of claim 4, the system software further comprising instructions for the processor to receive next step input manually input by a provider using a provider input device logged into one of the provider accounts, the next step input including the diagnosis, the next step, and the risk level status, the risk level status comprising an initial risk level manually input by the provider.

6. The computer-implemented healthcare management system of claim 1, the provider dashboard views comprising at least one batch processing control, the software further comprising instructions for the processor, in response to input from the provider input device selecting a plurality of patients in the patient table view and then activating the batch processing control, to execute a batch operation on all of the selected patients.

7. The computer-implemented healthcare management system of claim 6 wherein the batch operation comprises transmitting a message from one of the providers to another of the providers designating the selected patients.

8. The computer-implemented healthcare management system of claim 1, the patient table view comprising at least one sort control corresponding to a patient data value selected from the patient data, the software further comprising instructions for the processor, in response to the sort control being activated by input from a provider device logged into one of the provider accounts, to cause the patients displayed in the patient table view to be sorted by the patient data value, so that patients having the same value of the patient data value are displayed consecutively.

9. The computer-implemented healthcare management system of claim 1, the software further comprising instructions for the processor to cause a patient interface device to receive an automated notification at a preset time.

10. The computer-implemented healthcare management system of claim 9, the preset times being defined with respect to the agreed-upon due date of the next step.

11. The computer implemented healthcare management system of claim 9 wherein the automated notification comprises displaying a prompt for patient input on the patient display, the software further comprising instructions for the processor to receive patient input via the patient input device in response to the prompt and to schedule a follow-up automated notification for a later preset time determined by the patient input.

12. The computer implemented healthcare management system of claim 9, the software further comprising instructions for the processor to receive and store in the memory device scheduled patient appointment information from a provider input device, the scheduled patient appointment information including scheduled appointment times, wherein the automated notification comprises a guided self-advocacy prompt, the preset time being selected so that the prompt will be displayed when the patient is predicted to be at a scheduled appointment with a provider based on one of the stored scheduled appointment times, the prompt including instructions for the patient to ask the provider one or more questions and to enter the provider's responses before the conclusion of the appointment.
